SEQUENCE LISTING (43 sequences)

SEQ ID NO: 1 - DNA, 23 bp, Gallus gallus
tcacccgcagcagatcgccgcgg

SEQ ID NO: 2 - DNA, 23 bp, Gallus gallus
cgggtgaaggagctactggacgg

SEQ ID NO: 3 - DNA, 23 bp, Gallus gallus
gcaccacgcctgctgctccgggg

SEQ ID NO: 4 - DNA, 23 bp, Gallus gallus
gctgggctcccctcgcgggtcgg

SEQ ID NO: 5 - DNA, 23 bp, Gallus gallus
cctcgtgtctgtgggcagcgggg

SEQ ID NO: 6 - DNA, 5816 bp, Artificial Sequence
  Synthetic construct
  enhancer (1)..(293): human CMV IE1
  promoter (294)..(573): Chicken Beta Actin Promoter
  Intron (576)..(1546): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcttgatcgaagccgtctcaggggagagaccgcaggagcccgggctgggcataaaagtcagggcagagccatctattgcttacatttgcttctgacacaactgtgttcactagcaacctcaaacagacaccatggagcgcggggctcagccgggagtcggtgtgcggcggctccgcaatgtagcgcgggaggagcccttcgccgcggtcctgggcgcgctgcggggctgctacgccgaggccacgccgctggaggccttcgtccggcggctgcaggagggtggcaccggggaggtcgaggtgctgcgaggcgacgacgctcagtgctaccggaccttcgtgtcgcagtgcgtggtgtgcgtcccccgcggtgctcgcgccatcccccggcccatctgcttccagcagttatccagtcagagcgaagtcatcacaagaatcgttcagaggctgtgtgaaaagaaaaagaagaacatccttgcgtatggatactccttgctggatgagaacagttgtcacttcagagttttgccatcttcgtgtatatacagctatctgtccaatactgtaacagaaacgattcgcatcagtggcctctgggagatactgctgagtaggataggggacgacgtgatgatgtacctgctggagcactgtgcactcttcatgctggttcccccaagtaactgttaccaggtctgcgggcaaccaatttatgaacttatttcgcgtaacgtagggccatccccagggtttgttagacgacggtactcaaggtttaaacataatagcttgcttgactatgtgcgaaaaaggcttgtgtttcacaggcactatctttccaagtcgcagtggtggaagtgcaggccgagacgtcgaggtcgtgtctccagcaggagaaaaagaaggagccataggatacaaagcctaaggtctggttatcagccttctgcaaaagtgaactttcaagcaggtaggcagatcagcacagttactgcacgtctggaaaaacagagctgctccagtttatgtttgccagctagagcaccatctttaaaaaggaagcgtgatggagaacaggttgaaatcacagctaagagagtgaaaataatggagaaagagatagaggaacaggcttgtagtatcgttcctgatgtaaaccaaagtagctcccagaggcatggaacctcctggcatgtagcaccacgtgctgtaggtcttattaaagaacattacatttctgaaagaagtaacagtgagatgtctggtccttctgtagttcacagatctcaccctgggaagaggcctgtggcagacaaaagctcttttccacaaggagttcagggtaacaaacgcataaagaccggtgcagaaaaacgagcagaatccaatagaaggggcatagagatgtatataaacccaatccataaacccaatagaaggggcatagagaggcgtataaatccaacccacaaacctgagttgaattctgtacaaactgaaccaatggaaggtgcttcttcaggggacagaaagcaggaaaatcccccagctcatttggcaaagcagttaccaaatacattgtcgcgctctacagtgtactttgagaagaaatttcttctgtattcccgcagttaccaagaatattttcctaaatcgttcatactgagccgcctgcagggttgtcaggcaggtggaaggcggcttatagaaactatattcttaagccaaaacccattaaaggaacagcagaaccaaagcctaccacagcaaaagtggcgaaagaagaggttgcccaaacgctactggcaaatgagagagatatttcagaagctggtaaagaaccatgagaagtgcccttatttagttttcttgaggaaaaattgccctgttttgctttctgaagcatgtttgaaaaagacggagctgaccttgcaggcggctctgcctggggaagcaaaggttcacaagcacacagaacatgggaaagagtccactgagggtactgcaccgaacagcttcctcgctcctccctcagtgctagcatgtgggcagccagagagaggggaacagcaccctgcagaggggagtgatccgctcctcagggagctgctcaggcagcacagcagccactggcaggtgtatggctttgtgagggagtgcctggagcgggtgatccctgctgagctgtggggttcaagccataacaaatgccggttctttaaaaacgtgaaagcattcatttccatggggaagtatgctaagctttcattgcagcagctgatgtggaagatgagagtgaatgactgcgtatggcttcgtctggccaaaggtaatcactctgttcctgcctatgaacattgttaccgtgaagaaattctggcaaaattcctatactggctgatggattcctatgttatcgagttgctcaaatcatttttctatatcaccgagaccatgttccagaaaaacatgcttttctactaccgaaagtttatctggggcaagttacagaacattggaattagagaccattttgccaaagtacatctacgtgccttgtcttcagaggagatggaagtgatccgtcaaaaaaagtattttcctattgcatcaaggctccggttcattcctaaaatgaatggtttaagacccgtagtaagactaagccgtgttgttgaaggacagaaactcagcaaggaaagcagagaaaagaagatacagcgctataacactcagctaaaaaatctatttagtgttttaaactatgaacgaactgtaaacaccagtatcattggctcttcagtattcgggagagatgatatctacaggaagtggaaggagtttgttacaaaggtttttgaatcaggtggtgaaatgcctcatttctactttgtaaagggtgatgtatccagagcttttgataccattcctcacaagaaacttgtggaagtgatatcacaggtcttgaaacctgagagccaaactgtctatggaataaggtggtatgcagtgattatgattaccccaactggaaaagccaggaaactctataagagacatgtttctactttcgaggattttattccagacatgaagcagtttgtgtccaagcttcaagagagaacttcattacgaaatgcaatagtagttgaacagtgcttaacttttaatgagaacagttccaccctgtttactttctttcttcaaatgttacataataacatcctggagattgggcacaggtactatatacagtgctctggaatcccacagggctccattttgtcaaccttactttgcagcttatgctacggagacatggaaaacaaattactctgtgggatccagaaggatggagtcctaatacgtcttattgatgactttttgctggttacgccacatttaatgcaggcaagaacttttctaaggactatagcagcaggtattcctgagtatggctttttaataaatgccaagaagactgtggtgaattttcctgttgatgatatcccgggatgttccaagttcaaacatctgccagattgtcgtttgatctcatggtgtggtttattattggatgtgcagacacttgaggtttattgtgattactccagttatgcctttacttctatcagatcaagtctttccttcaattcaagtagaatagctgggaaaaacatgaaatgcaaattgactgcagtcctcaaactgaaatgccatcctttacttcttgacttaaagatcaacagccttcagacagttctaattaacatctacaagatatttttacttcaggcttacaggttccatgcctgtgttcttcagcttccattcaaccagaaagttaggaataatcctgatttcttcctaaggatcatctctgatactgcttcatgctgctattttatcctgaaagctaaaaatccaggagtttctttaggtagcaaagatgcatctggcatgttcccttttgaggcagcagaatggctgtgctaccatgccttcattgtcaaactgtccaaccacaaagttatttacaaatgcttacttaagccccttaaagtctataagatgcatctgtttgggaagatcccaagggatactatggaactgctgaagacggtgacggaaccatcgctttgtcaagatttcaaaactatactggactaa

SEQ ID NO: 7 - DNA, 5854 bp, Artificial Sequence
  Synthetic construct
  enhancer (1)..(293): human CMV IE1
  promoter (294)..(575): Chicken Beta Actin Promoter
  Intron (578)..(1548): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcttgatcgaagccgtctcaggggagagaccgcaggagcccgggctgggcataaaagtcagggcagagccatctattgcttacatttgcttctgacacaactgtgttcactagcaacctcaaacagacaccatggagcgcggggctcagccgggagtcggtgtgcggcggctccgcaatgtagcgcgggaggagcccttcgccgcggtcctgggcgcgctgcggggctgctacgccgaggccacgccgctggaggccttcgtccggcggctgcaggagggtggcaccggggaggtcgaggtgctgcgaggcgacgacgctcagtgctaccggaccttcgtgtcgcagtgcgtggtgtgcgtcccccgcggtgctcgcgccatcccccggcccatctgcttccagcagttatccagtcagagcgaagtcatcacaagaatcgttcagaggctgtgtgaaaagaaaaagaagaacatccttgcgtatggatactccttgctggatgagaacagttgtcacttcagagttttgccatcttcgtgtatatacagctatctgtccaatactgtaacagaaacgattcgcatcagtggcctctgggagatactgctgagtaggataggggacgacgtgatgatgtacctgctggagcactgtgcactcttcatgctggttcccccaagtaactgttaccaggtctgcgggcaaccaatttatgaacttatttcgcgtaacgtagggccatccccagggtttgttagacgacggtactcaaggtttaaacataatagcttgcttgactatgtgcgaaaaaggcttgtgtttcacaggcactatctttccaagtcgcagtggtggaagtgcaggccgagacgtcgaggtcgtgtctccagcaggagaaaaagaaggagccataggatacaaagcctaaggtctggttatcagccttctgcaaaagtgaactttcaagcaggtaggcagatcagcacagttactgcacgtctggaaaaacagagctgctccagtttatgtttgccagctagagcaccatctttaaaaaggaagcgtgatggagaacaggttgaaatcacagctaagagagtgaaaataatggagaaagagatagaggaacaggcttgtagtatcgttcctgatgtaaaccaaagtagctcccagaggcatggaacctcctggcatgtagcaccacgtgctgtaggtcttattaaagaacattacatttctgaaagaagtaacagtgagatgtctggtccttctgtagttcacagatctcaccctgggaagaggcctgtggcagacaaaagctcttttccacaaggagttcagggtaacaaacgcataaagaccggtgcagaaaaacgagcagaatccaatagaaggggcatagagatgtatataaacccaatccataaacccaatagaaggggcatagagaggcgtataaatccaacccacaaacctgagttgaattctgtacaaactgaaccaatggaaggtgcttcttcaggggacagaaagcaggaaaatcccccagctcatttggcaaagcagttaccaaatacattgtcgcgctctacagtgtactttgagaagaaatttcttctgtattcccgcagttaccaagaatattttcctaaatcgttcatactgagccgcctgcagggttgtcaggcaggtggaaggcggcttatagaaactatattcttaagccaaaacccattaaaggaacagcagaaccaaagcctaccacagcaaaagtggcgaaagaagaggttgcccaaacgctactggcaaatgagagagatatttcagaagctggtaaagaaccatgagaagtgcccttatttagttttcttgaggaaaaattgccctgttttgctttctgaagcatgtttgaaaaagacggagctgaccttgcaggcggctctgcctggggaagcaaaggttcacaagcacacagaacatgggaaagagtccactgagggtactgcaccgaacagcttcctcgctcctccctcagtgctagcatgtgggcagccagagagaggggaacagcaccctgcagaggggagtgatccgctcctcagggagctgctcaggcagcacagcagccactggcaggtgtatggctttgtgagggagtgcctggagcgggtgatccctgctgagctgtggggttcaagccataacaaatgccggttctttaaaaacgtgaaagcattcatttccatggggaagtatgctaagctttcattgcagcagctgatgtggaagatgagagtgaatgactgcgtatggcttcgtctggccaaaggtaatcactctgttcctgcctatgaacattgttaccgtgaagaaattctggcaaaattcctatactggctgatggattcctatgttatcgagttgctcaaatcatttttctatatcaccgagaccatgttccagaaaaacatgcttttctactaccgaaagtttatctggggcaagttacagaacattggaattagagaccattttgccaaagtacatctacgtgccttgtcttcagaggagatggaagtgatccgtcaaaaaaagtattttcctattgcatcaaggctccggttcattcctaaaatgaatggtttaagacccgtagtaagactaagccgtgttgttgaaggacagaaactcagcaaggaaagcagagaaaagaagatacagcgctataacactcagctaaaaaatctatttagtgttttaaactatgaacgaactgtaaacaccagtatcattggctcttcagtattcgggagagatgatatctacaggaagtggaaggagtttgttacaaaggtttttgaatcaggtggtgaaatgcctcatttctactttgtaaagggtgatgtatccagagcttttgataccattcctcacaagaaacttgtggaagtgatatcacaggtcttgaaacctgagagccaaactgtctatggaataaggtggtatgcagtgattatgattaccccaactggaaaagccaggaaactctataagagacatgtttctactttcgaggattttattccagacatgaagcagtttgtgtccaagcttcaagagagaacttcattacgaaatgcaatagtagttgaacagagatttttactaaactgttacagtctaattcttcagtgcttaacttttaatgagaacagttccaccctgtttactttctttcttcaaatgttacataataacatcctggagattgggcacaggtactatatacagtgctctggaatcccacagggctccattttgtcaaccttactttgcagcttatgctacggagacatggaaaacaaattactctgtgggatccagaaggatggagtcctaatacgtcttattgatgactttttgctggttacgccacatttaatgcaggcaagaacttttctaaggactatagcagcaggtattcctgagtatggctttttaataaatgccaagaagactgtggtgaattttcctgttgatgatatcccgggatgttccaagttcaaacatctgccagattgtcgtttgatctcatggtgtggtttattattggatgtgcagacacttgaggtttattgtgattactccagttatgcctttacttctatcagatcaagtctttccttcaattcaagtagaatagctgggaaaaacatgaaatgcaaattgactgcagtcctcaaactgaaatgccatcctttacttcttgacttaaagatcaacagccttcagacagttctaattaacatctacaagatatttttacttcaggcttacaggttccatgcctgtgttcttcagcttccattcaaccagaaagttaggaataatcctgatttcttcctaaggatcatctctgatactgcttcatgctgctattttatcctgaaagctaaaaatccaggagtttctttaggtagcaaagatgcatctggcatgttcccttttgaggcagcagaatggctgtgctaccatgccttcattgtcaaactgtccaaccacaaagttatttacaaatgcttacttaagccccttaaagtctataagatgcatctgtttgggaagatcccaagggatactatggaactgctgaagacggtgacggaaccatcgctttgtcaagatttcaaaactatactggactaa

SEQ ID NO: 8 - DNA, 23 bp, Bos taurus
ggcggccaacaagtcggccgagg

SEQ ID NO: 9 - DNA, 23 bp, Bos taurus
gccaacgcgccgaaccgttacgg

SEQ ID NO: 10 - DNA, 23 bp, Bos taurus
cctcgggtgcaaagactccgcgg

SEQ ID NO: 11 - DNA, 4993 bp, Artificial Sequence
  Synthetic construct
  enhancer (1)..(293): human CMV IE1
  promoter (294)..(575): Chicken Beta Actin Promoter
  Intron (578)..(1548): Intron Chicken beta actin
  ref <308>: DATABASE ACCESSION NUMBER: NCBI RefSeq / NM_001046242.1
  ref <309>: DATABASE ENTRY DATE: 2016-04-02
  ref <313>: RELEVANT RESIDUES IN SEQ ID NO: (1646)..(4993)
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgccgcgcgcgcccaggtgccgggccgtgcgcgcccttctgcgggccagctaccggcaggtgctgcccctggccgccttcgtacggcgcctgcggccccagggccaccggcttgtgcggcgcggggacccggcggccttccgcgcgctggtggctcagtgcttggtgtgcgtgccctgggacgcgcagccgccccctgccgccccgtccttccgccaggtgtcctgcctgaaggagctggtggccagagtcgtgcagaggctctgcgagcgcggcgcgaggaacgtgctggccttcggcttcacgctgctggccggggcccgcggcgggccgcccgtggccttcacgaccagcgtacgcagctacctgcccaacacggtaaccgacacgctgcgcggcagcggcgcctgggggctgctgctgcaccgcgtgggcgacgacgtgctcacccacctgctgtcgcgctgcgcgctctacctgctggtgcccccgacctgcgcctaccaggtgtgtgggccgccgctctatgacctccgcgccgccgccgccgccgctcgtcggcccacgcggcaagtgggcgggacccgggcgggcttcggactcccgcgcccggcctcgtcgaacggcggccacggggaggccgaaggactcctggaggcgcgggcccagggcgcgaggcggcgtcgcagtagcgcgcggggacgactgcctccagccaagaggcccaggcgcggcctggagcccgggcgggatctcgaagggcaggtggcccgcagcccgccccgcgtggtgacacctacccgagacgctgcggaagccaagtctcggaagggcgacgtgcccgggccctgccgcctcttcccgggcggcgagcggggtgtcggctccgcgtcctggcggctgtcaccctcggagggcgagccgggtgccggagcttgcgctgagaccaagaggttcctttactgctccggcggtggcgaacagctgcgccgctccttcctgctctgctccctgcctcccagcctggccggggcgcggacactcgtggaaaccatctttctggactcgaagcccgggccgccaggggctccccgccggccgcgccgcctgcccgcgcgctactggcagatgcggcccctgttccggaaactgcttgggaaccacgcgcggtgcccctatggcgcgctgctcagggcgcactgcccgctgccggcctctgcgccccgggcggggccagaccatcagaagtgccctggtgttgggggctgcccctctgagaggccggccgctgcccccgagggcgaggcgaactcagggcgcctggtccagctgctccgccagcacagcagcccctggcaggtgtacggcctcctgcgggcctgtcttcgccgcctggtgcccgccggcctctggggctcccggcacaacgagcggcgcttcctgcggaacgtgaagaagctcctctccctggggaagcacggcaggctctcgcagcaggagctcacgtggaagatgaaggtgcaggactgcgcctggctgcgcgcgagcccaggggctcgctgcgtgcccgccgcggagcaccgccagcgcgaggccgtcctgggtcgcttcctgcactggctgatgggcgcctacgtggtggagctgctcaggagcttcttctacgtcacagagaccacgttccagaagaaccggctcttcttcttccggaagcgcatctggagccagctgcagcgcctgggcgtcagacaacacttagaccgtgtgcggcttcgagaactgtcagaagcagaggtcaggcagcaccaggaggccaggccggctctgctgacatccaggctccgtttcgtccccaagcccggcgggctgcggcccatcgtgaacgtgggctgtgttgagggcgccccggcaccgcccagagacaagaaggtgcagcatctcagctcacgggtcaagacgctgttcgcggtgctgaactacgagcgagctcggcggcctggcctcctgggggcctcggtgctgggcatggacgacatccacagggcctggcgggccttcgtgctgcccctgagggcccggggcccagcccccccgctctacttcgtcaaggtggacgtggtgggggcctacgatgccctcccccaggataagctggcagaggtgatcgctaacgtgctgcagccgcaggagaatacgtactgcgtgcgccactgcgccatggtccggactgcgcgcgggcgcatgcgcaagtccttcaagagacacgtgtccaccttctcggacttccagccgtacctgaggcagctcgtggagcatctgcaggcgatgggctccctgagggacgccgtggtcatcgagcagagctgctccctgaacgagcctggcagcagcctcttcaacctcttcctgcacctgaggtcctacatccagtgtcaggggatcccccagggctccatcctgtccaccctgctctgcagcttctgctatggggacatggagaacaagctcttccctggagtccagcaggacggggtgcttctgcgcctggtggacgacttcctgctggtcaccccgcacctgacgcgggccagagacttcctcaggacgctggtgcgcggtgtgcctgagtatggctgccaggtgaacctgcggaagacggtggtgaacttccccgtggagcccggggccctgggcggcgcggcgcccctgcagctgccggcccactgcctgttcccctggtgcggcctgctgctggatacccgcaccctggaggtgcatggcgaccactccagttacgcccggacgtccatcagagcgagtctcaccttcacccagggcttcaagcccgggaggaacatgcgtcgcaagctgttggcggtcttgcagctcaagtgccatgggctcttcctggacctgcaggtgaacagtctgcagacggtcttcacaaacgtttacaagatattcctgctgcaggcctacaggttccacgcctgcgtgctgcagctgcccttcagccagccggtcaggagcagccccgcgttctttctccaggtcatcgccgacaccgcatcccgcggctacgccctcctgaaagccaggaacgcaggggcgtcactgggggccaggggcgccgccggcctgttcccgtctgaagctgcgcagtggctgtgtctccacgccttcctgctcaagctggctcaccaccgtgtcacctacagccgcctgctgggggccctccggacagcccaagcacggctgcaccggcagctcccggggcccacacgggccgccctggaggcggcggccgaccccgccctgaccgcagacttcaagaccatcttggactga

SEQ ID NO: 12 - DNA, 2557 bp, Artificial Sequence
  Synthetic construct
  enhancer (1)..(293): human CMV IE1
  promoter (294)..(575): Chicken Beta Actin Promoter
  Intron (578)..(1548): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggctacctcccgatatgagccagtggctgagattggtgtcggtgcctatgggacagtgtacaaggcccgtgatccccacagtggccactttgtggccctcaagagtgtaagagtccccaatggaggaggtgctggagggggcctgcccatcagcaccgttcgggaggtggccttactgcggcgtctggaggcttttgagcatcccaatgttgtcaggcttatggacgtctgtgcaacagcccgaactgaccgggagaccaaagtgaccctggtgtttgagcatgtggaccaagatctcaggacatatctggacaaggcacccccaccaggcttgccagtggagaccataaaagatctgatgcgccaatttctaagaggcctggatttccttcatgccaactgcatcgttcaccgagacctgaagccagagaacattctggtgacaagtggtgggacagtcaagctggctgactttggcctggccagaatctacagctaccagatggcacttacacctgtggttgttacactctggtatcgtgctccagaagttcttttgcagtctacgtatgcaacacccgtggacatgtggagcgttggctgtatctttgcagagatgtttcgtcgaaagcctctcttctgtggaaactctgaagctgaccagttaggcaaaatctttgacctgattggactgcccccagaggatgactggccccgagatgtctctctaccccgaggagccttttccccccgagggccccgcccagtgcagtcggtggtccctgagctggaggaatctggagcacagctgctgctggagatgctgacttttaacccacacaagcgaatctctgccttccgagccctgcagcactcttatctacacaaggcagaaggtgacgcagagtga

SEQ ID NO: 13 - DNA, 2557 bp, Artificial Sequence
  Synthetic Construct
  enhancer (1)..(293): human CMV IE1
  promoter (294)..(575): Chicken Beta Actin Promoter
  Intron (578)..(1548): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggctacctcccgatatgagccagtggctgagattggtgtcggtgcctatgggacagtgtacaaggcctgtgatccccacagtggccactttgtggccctcaagagtgtaagagtccccaatggaggaggtgctggagggggcctgcccatcagcaccgttcgggaggtggccttactgcggcgtctggaggcttttgagcatcccaatgttgtcaggcttatggacgtctgtgcaacagcccgaactgaccgggagaccaaagtgaccctggtgtttgagcatgtggaccaagatctcaggacatatctggacaaggcacccccaccaggcttgccagtggagaccataaaagatctgatgcgccaatttctaagaggcctggatttccttcatgccaactgcatcgttcaccgagacctgaagccagagaacattctggtgacaagtggtgggacagtcaagctggctgactttggcctggccagaatctacagctaccagatggcacttacacctgtggttgttacactctggtatcgtgctccagaagttcttttgcagtctacgtatgcaacacccgtggacatgtggagcgttggctgtatctttgcagagatgtttcgtcgaaagcctctcttctgtggaaactctgaagctgaccagttaggcaaaatctttgacctgattggactgcccccagaggatgactggccccgagatgtctctctaccccgaggagccttttccccccgagggccccgcccagtgcagtcggtggtccctgagctggaggaatctggagcacagctgctgctggagatgctgacttttaacccacacaagcgaatctctgccttccgagccctgcagcactcttatctacacaaggcagaaggtgacgcagagtga

SEQ ID NO: 14 - DNA, 2559 bp, Artificial Sequence
  Synthetic construct
  enhancer (1)..(293): human CMV IE1
  promoter (294)..(575): Chicken Beta Actin Promoter
  Intron (578)..(1548): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggctacctctcgatatgagccagtggctgaaattggtgtcggtgcctatgggacagtgtacaaggcctgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgaaa

SEQ ID NO: 15 - DNA, 2542 bp, Artificial Sequence
  Synthetic Construct
  enhancer (1)..(293): human CMV IE1
  promoter (294)..(575): Chicken Beta Actin Promoter
  Intron (578)..(1548): Intron Chicken beta actin
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagaacttccagaaggtggagaagatcggggagggcacctatggtgtggtgtacaaggctcgcaacaagcgcacgggcgagctggtggcgctcaagaagatccggctggacacggagaccgagggtgtccccagcactgccatccgagaaatctcgctgctgaaggagctgaagcatcccaacatagtcaaactgctggacgtgatccacacggagaacaagctctacctggtctttgagttcctgcaccaggacctgaagaagttcatggatgcatcatccctgggcggcatcgcgctgcccctcatcaagagctacctgttccagctgctgcaaggcctggccttctgccacgcacaccgcgtgctgcaccgtgacctcaaaccccagaacctcctcatcaacgccgacggtgccatcaagctggctgacttcgggctggcccgcgcctttggggtgcccgtgcgcacctacacacacgaggtggtgacgttgtggtaccgtgcgcctgagatcctgctgggctgcaagtactattcgactgctgtggacatctggagcctgggctgcatttttgctgagatggtgacgcggcgcgcgctcttccccggggattcggagatcgatcagctcttccgtatcttccgcacgttggggacaccggatgaggccgcctggcccggcgtcaccgcgctgcccgactacaagcccagcttccccaaatgggcccggcaggatctgggcaaggtggtgccgccgctggatgaggagggccgcaagctgctggctcaaatgctgcactacgatcccaacaagcgcatctcggccaaggcagcgctgagccaccccttcttccgcgacgtcaccagggctgtcccccacctgcgcctgtga

SEQ ID NO: 16 - DNA, 2626 bp, Artificial Sequence
  Synthetic construct
  enhancer (1)..(293): human CMV IE1
  promoter (294)..(575): Chicken Beta Actin Promoter
  Intron (578)..(1548)
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggacaaggacggcaccaacctggccgaccagcagtatgagtgcgtggctgagatcggcgagggagcctacgggaaggtgttcaaggcccgcgacctgaagaatggcggccgcttcgtggcgctgaagcgggtgcgggtgcagaccagcgaggagggcatgccgctgtccaccatccgggaggtggccgtcctgaggcacctggagaccttcgagcaccccaacgtggtcagattgtttgatgtgtgcaccgtgtcacgaacagacagagaaaccaagttaacgttggtgtttgaacatgtggatcaagacttgactacttacttggataaagttccagagcctggagtgcctactgaaactataaaggatatgatgcttcagctgtttcggggactggattttctgcattcacatcgtgtggtgcatcgggacctgaaaccccagaatatccttgtaaccagcagtgggcagataaagttagctgactttggacttgcacgaatctacagttttcagatggctcttacatcagtggttgttactttgtggtatagagctcctgaagttttgcttcagtccagctatgcaacaccagttgatctttggagtgttggttgcatatttgcagaaatgttccgtcgaaaaccactcttccgtggaaattcagatgttgatcagctaggaaaaatctttgatgtaattggactcccagaagaagaggactggcctaatgatgtggcccttccaagaaatgcttttgcttccagacccgcacaacctattgaaaaatttgtaccagatattgatgacatgggcaaagacttgcttcttaaatgcttagcgttcaatccagccaagagaatatctgcctatgctgccctgtctcacccctatttccatgatctggagaaatgcaaggagaatctggactctcacatgtcatccagccaaaactccagtgaggtgaacgcatcataa

SEQ ID NO: 17 - protein, 168 aa, Mus musculus
  ref <308>: DATABASE ACCESSION NUMBER: NCBI RefSeq / NP_001035744.1
  ref <309>: DATABASE ENTRY DATE: 2015-05-12
  ref <313>: RELEVANT RESIDUES IN SEQ ID NO: (1)..(168)
MESAADRLARAAAQGRVHDVRALLEAGVSPNAPNSFGRTPIQVMMMGNVHVAALLLNYGADSNCEDPTTFSRPVHDAAREGFLDTLVVLHGSGARLDVRDAWGRLPLDLAQERGHQDIVRYLRSAGCSLCSAGWSLCTAGNVAQTDGHSFSSSTPRALELRGQSQEQS

SEQ ID NO: 18 - protein, 152 aa, Bos taurus
  ref <308>: DATABASE ACCESSION NUMBER: NCBI RefSeq / XP_010806061.1
  ref <309>: DATABASE ENTRY DATE: 2016-01-26
  ref <313>: RELEVANT RESIDUES IN SEQ ID NO: (1)..(152)
METSADLLAAAAALGWAEEVRALLEAGASANAPNRYGRSAIQVMMMGSARVAELLLLHGADPNCADPATLTRPVHDAAREGFLDTLVALHRAGARLDVRDAWGRLPVDLAEERGHRDVARYLRAAAEDTEGGSHASADSAEGPADSSDLKKD

SEQ ID NO: 19 - protein, 152 aa, Sus scrofa
  ref <308>: DATABASE ACCESSION NUMBER: GenBank / CAC87046.1
  ref <309>: DATABASE ENTRY DATE: 2005-04-15
  ref <313>: RELEVANT RESIDUES IN SEQ ID NO: (1)..(152)
MEPSADWLASAAARGREGEVRALLEAGALANAPNRYGRTPIQVMMMGSTRVAELLLLHGADPNCEDPATLTRPVHDAAREGFLDTLVVLHRAGARLDVRDAWGRLPVDLAEERGHRDVAGYLRANAGRTEGGSHARSNSGEDPADISNLQNH

SEQ ID NO: 20 - protein, 139 aa, Gallus gallus
  ref <308>: DATABASE ACCESSION NUMBER: NCBI RefSeq / NP_989764.1
  ref <309>: DATABASE ENTRY DATE: 2016-11-06
  ref <313>: RELEVANT RESIDUES IN SEQ ID NO: (1)..(139)
MAQRAASTAADELANAAARGDLLRVKELLDGAADPNAVNSFGRTPIQVMMLGSPRVAELLLQRGADPNRPDPRTGCRPAHDAARAGFLDTLAALHRAGARLDLPDGRGRLPIDVAAGGPHGPVGCYLRRLPALPRAPLP

SEQ ID NO: 21 - protein, 130 aa, Mus musculus
  ref <308>: DATABASE ACCESSION NUMBER: NCBI RefSeq / NP_031696.1
  ref <309>: DATABASE ENTRY DATE: 2015-02-15
  ref <313>: RELEVANT RESIDUES IN SEQ ID NO: (1)..(130)
MLGGSSDAGLATAAARGQVETVRQLLEAGADPNALNRFGRRPIQVMMMGSAQVAELLLLHGAEPNCADPATLTRPVHDAAREGFLDTLVVLHRAGARLDVCDAWGRLPVDLAEEQGHRDIARYLHAATGD

SEQ ID NO: 22 - protein, 131 aa, Sus scrofa
  ref <308>: DATABASE ACCESSION NUMBER: NCBI RefSeq / NP_999289.1
  ref <309>: DATABASE ENTRY DATE: 2016-07-29
  ref <313>: RELEVANT RESIDUES IN SEQ ID NO: (1)..(131)
MLSGGGGDAGLANAAARGQVETVRQLLEAGADPNGLNHFGRRPIQVMMMGSARVAELLLLHGADPNCADPATLTRPVHDAAREGFLDTLVALRRAGARLDVQDAWGRLPVDLAEERGHRDVARFLRAAAGD

SEQ ID NO: 23 - protein, 131 aa, Bos taurus
  ref <308>: DATABASE ACCESSION NUMBER: NCBI RefSeq / NP_001069362.1
  ref <309>: DATABASE ENTRY DATE: 2016-07-29
  ref <313>: RELEVANT RESIDUES IN SEQ ID NO: (1)..(131)
MLSGGGGDADLANAAARGQVEAVRQLLEAGVDPNRLNRFGRRPIQVMMMGSARVAELLLLHGADPNCADPATLTRPVHDAAREGFLDTLVALHRAGGRLDVRDAWGRLPVDLAEERGHRDVARYLRATAGD

SEQ ID NO: 24 - protein, 126 aa, Oncorhynchus mykiss
  ref <308>: DATABASE ACCESSION NUMBER: GenBank / ACO08670.1
  ref <309>: DATABASE ENTRY DATE: 2009-03-27
  ref <313>: RELEVANT RESIDUES IN SEQ ID NO: (1)..(126)
MTMPLEDDLASAAATGNTNRVKILLQSGVDVNGVNCFGRTPLQVMMMGGSPVAQLLLMQGADPNIADRHTGTTPLHDAARMGFLDTVEILVQFLADPNSRDNRNCRPIDLAIESGHNNVVAFLKAL

SEQ ID NO: 25 - protein, 127 aa, Oreochromis niloticus
  ref <308>: DATABASE ACCESSION NUMBER: NCBI RefSeq / XP_003452283.1
  ref <309>: DATABASE ENTRY DATE: 2016-12-05
  ref <313>: RELEVANT RESIDUES IN SEQ ID NO: (1)..(127)
MTLQDELTTAAAKGNTAAVKALLDRGAQVNGTNSFGRTALQVMMMGSTSVAQLLLEHGANPNVGDSSTGASPLHDAARTGFVDTVHLLVQHHADPQARDKLNRLPVDLARQHGHGDVVDFLESLQNP

SEQ ID NO: 26 - DNA, 20 bp, Gallus gallus
ctctccgtcctccctacctg

SEQ ID NO: 27 - DNA, 20 bp, Gallus gallus
gtaccaactgcggggagaaa

SEQ ID NO: 28 - DNA, 20 bp, Gallus gallus
ggacgccggtcaatgaatca

SEQ ID NO: 29 - DNA, 20 bp, Gallus gallus
caggtgatgatgctgggcag

SEQ ID NO: 30 - DNA, 20 bp, Gallus gallus
tttctccccgcagttggtac

SEQ ID NO: 31 - DNA, 20 bp, Gallus gallus
ctgcaagacccaagacgtct

SEQ ID NO: 32 - DNA, 20 bp, Bos taurus
gcctagtcccacaccctttc

SEQ ID NO: 33 - DNA, 20 bp, Bos taurus
catttaagcctggcccctga

SEQ ID NO: 34 - DNA, 20 bp, Bos taurus
tgtccgactctttgccatcc

SEQ ID NO: 35 - DNA, 20 bp, Bos taurus
gaccctggataaggcgtcag

SEQ ID NO: 36 - DNA, 20 bp, Bos taurus
agtgaatgctctgggaagcg

SEQ ID NO: 37 - DNA, 20 bp, Bos taurus
gattgtcagcgcatctgcag

SEQ ID NO: 38 - DNA, 20 bp, Bos taurus
tagagatctgaaccccacgc

SEQ ID NO: 39 - DNA, 20 bp, Bos taurus
ctctgatgggagtggggaga

SEQ ID NO: 40 - DNA, 20 bp, Bos taurus
aggcctttcctacctggtct

SEQ ID NO: 41 - DNA, 20 bp, Bos taurus
taattccgctggtttcccaa

SEQ ID NO: 42 - DNA, 20 bp, Bos taurus
aaactgctgcgacatctgga

SEQ ID NO: 43 - DNA, 20 bp, Bos taurus
acggtccctcttctctctcc

(19) United States
(12) Patent Application Publication
(10) Pub. No.: US 2019/0024079 A1
(43) Pub. Date: Jan. 24, 2019

(54) METHODS FOR EXTENDING THE REPLICATIVE CAPACITY OF SOMATIC CELLS DURING AN EX VIVO CULTIVATION PROCESS

(71) Applicant: Memphis Meats, Inc., Berkeley, CA (US)

(72) Inventors: Nicholas J. GENOVESE, Hayward, CA (US); Danielle Nicole DESMET, Berkeley, CA (US); Eric SCHULZE, San Francisco, CA (US)

(21) Appl. No.: 16/070,251

(22) PCT Filed: Jan. 17, 2017
(86) PCT No.: PCT/US2017/013782

§ 371 Date: Jul. 13, 2018

Related U.S. Application Data
(60) Provisional application No. 62/361,867, filed on Jul. 13, 2016.
(60) Provisional application No. 62/278,869, filed on Jan. 14, 2016.

(51) Int. Cl. C12N 15/11; C12N 9/22; C12N 5/077; C12N 9/12; A23L 33/135

(52) U.S. Cl. CPC C12N 15/11; C12N 9/22; C12N 5/0658; C12N 9/12; C12Y 207/11022; A23V 2002/00; C12N 2310/20; C12N 2800/80; C12N 2523/00; C12N 2510/02; C12N 2511/00; A23L 33/135

(57) ABSTRACT

A product and process for extending the replicative capacity of metazoan somatic cells using targeted genetic amendments to abrogate inhibition of cell-cycle progression during replicative senescence and derive clonal cell lines for scalable applications and industrial production of metazoan cell biomass. An insertion or deletion mutation using guide RNAs targeting the first exon of the transcript encoding each protein is created using CRISPR/Cas9. Targeted amendments result in inactivation of p15 and p16 proteins which increases the proliferative capacity of the modified cell populations relative to their unaltered parental populations. Combining these amendments with ancillary telomerase activity from a genetic construct directing expression of a telomerase protein homolog from a TERT gene, increases the replicative capacity of the modified cell populations indefinitely. One application is to manufacture skeletal muscle for dietary consumption using cells from the poultry species Gallus gallus; another is from the livestock species Bos taurus.

### TECHNICAL FIELD OF THE INVENTION

The present invention relates to a product and process that extends the replicative capacity of metazoan cells for scalable manufacturing of biomass in industrial bioprocess applications.

### BACKGROUND OF THE INVENTION

Myogenic (i.e., “muscle forming”) cell lines have been used as fundamental models for understanding skeletal muscle biology since their derivation was first described nearly 50 years ago. Beyond basic research, muscle (i.e. myoblast) cell lines have prospective industrial applications in biological robotics; bioartificial muscle constructs screening pharmacological compounds; therapeutic correction of hereditary muscle disease; and the ex vivo production of edible biomass for dietary consumption. Primary muscle cell procurement from donor tissues as a cell source for applications in commercial-scale production of animal biomass requires technical and material resources that currently preclude application-specific batch cultivation. Moreover, the native capacity of primary cells to replicate limits the scale at which they can be passaged for genetic amendment or used for cell banking and industrial manufacturing. To address these challenges, this invention comprises genetic amendments which extend the renewal capacity of cells committed to the skeletal muscle lineage to create what is hereafter referred to as a “myogenic cell line”.

A fully functional cell line, myogenic or otherwise, is identified by a lineage-committed ontological phenotype, stability of the genome with a broader karyotype, a capacity for perpetual renewal and potential to exhibit functional features of terminally differentiated cells that exist in their histological counterparts in vivo. For a skeletal muscle cell line, some of these characteristics include cell fusion, striated myofibril development and physiological response to chemical stimuli such as acetylcholine.

Successes in the derivation of certain skeletal muscle cell lines have been attained through serial passage of isolates from normal tissues and mutant tissues, or isolated from tumors. To date, existing myogenic cell lines remain poorly characterized, often having undesirable traits such as genomic instability, aneuploidy, impaired differentiation capacity and pleotropic alteration of their molecular signaling and transcription networks.

Available options for immortalized cell lines remain limited to a select group of progenitor species. Most, if not all, of these cell lines have unfavorable features that potentially diminish their functional suitability for applications requiring species-specific or high-fidelity representation of a selected progenitor species. For example, the derivation of immortalized myogenic cell lines from agriculturally important animal species such as cattle, swine, chickens and salmon has not been achieved. The selection of existing myogenic cell lines is largely limited to model species commonly used in biomedical research, namely, murine and primate species; but these cell lines are not generally acceptable as a progenitor source to produce edible biomass for use in foods.

This is also true of other approaches used to establish myogenic cell lines by either directly targeting individual pathways that contribute to replicative senescence, such as the retinoblastoma family protein (i.e., p107, pRB, p130)-mediated inhibition of the cell cycle entry, and the DNA-damage response elicited by shortening of telomeric DNA sequences repeats (TTAGGG)ₙ at the ends of chromosomes during successive cell division cycles. For example, ablation of pRB function impairs the ability of myogenic cells to reach and maintain a terminally differentiated state and, alone, is insufficient to maintain their proliferative capacity. Likewise, long-term maintenance of telomerase activity by overexpression of functional telomere reverse transcriptase (“TERT”) extends the regenerative capacity of primary myoblasts by counteracting telomere erosion; alone it is insufficient to prevent senescence by these cells.

In normal skeletal muscle, p107 is the dominant retinoblastoma family protein expressed and complexed with E2F transcription factors in undifferentiated, proliferating myoblasts. During terminal differentiation, myoblasts exit the cell division cycle and fuse into multinucleated myotubes that mature into contractile muscle fibers. The roles of retinoblastoma family proteins pRB and p130 in repressing E2F-directed transcriptional activation of gene expression to initiate cell-cycle exit during myoblast differentiation and maintain a post-mitotic state in myotubes are directed by cyclin-dependent kinase inhibitors (“CKIs”). But in mammalian skeletal muscle modeled and characterized to date, the functions of CKIs in isolation are not sufficient to arrest the cell cycle during terminal differentiation. Rather, roles shared by CKIs in combination including p21, p18, p27 and p57 direct and stabilize exit from the cell cycle during myoblast differentiation. No consensus has been established as to which combination of CKIs are sufficient and necessary to effectively perform this role. Notwithstanding this conclusion, the CM proteins p15 and p16 have not been implicated in this role.

The CM protein p16 (also referred to as INK4A) is an expression product of the CDKN2A gene found in mammals which constitutes part of the INK4B-ARF-INK4A locus shared by sequential CDKN2B and CDKN2A genes. The CDKN2A gene encodes transcripts for two proteins, ARF and p16, which are translated from alternate splice variants. The expression of each CDKN2A transcript is under the regulation of a distinct promoter. Though the ARF and p16 proteins are encoded by shared exons within the CDKN2A gene, the ARF transcript is transcribed in an alternate reading frame relative to the p16 transcript, hence the amino acid sequences comprising ARF and p16 are entirely distinct. The CDKN2B gene encodes the p15 protein (also referred to as INK4B), which is a CKI similar to p16 in both structure and function.

P15 and p16 are paralogs present in mammals, wherein the CDKN2A gene is thought to have arisen from duplication of the CDKN2B gene coincident with the divergence of mammals within the metazoan phylogeny. Unlike p15, p16 acts as the dominant CKI in mammals during replicative senescence.

Notably, the naked mole rat, a long-lived tumor-resistant rodent, is known to express a p15/p16 hybrid protein from a transcriptional splice variant bridging the first exon of p15 with the second and third exons of p16. In fish and in birds, elements of the INK4B-ARF-INK4A locus are only partially conserved. In the chicken genome, the CDK2NA gene does not encode a p16 protein; only p15 and ARF proteins are encoded. Though sequenced and annotated fish genomes feature a CDKN2B gene, the CDKN2A gene is not present within these genomes. The chicken p15 protein corresponds functionally to the human p16 protein; both bind CDK4/6 and restrict cell cycle progression when overexpressed in human fibroblasts. Moreover, p15 may facilitate replicative senescence in chicken fibroblasts.

In addition, deletion mutations within the immortal myogenic murine C2C12 cell line, specifically in the INK4B-ARF-INK4A locus, abrogate the expression of p16 and ARF. Yet no myogenic cell lines reported to date have been generated by targeted, non-stochastic genetic amendment of the INK4B-ARF-INK4A locus. Rather, the repressive CKI function of the p16 protein in human myogenic cells has been partially surmounted by ectopic overexpression of the CDK4 (i.e. cyclin-dependent kinase 4) protein to overcome senescence and derive cell lines.

Other methods, such as short hairpin RNA (shRNA) silencing of p16 and p15 function have not been demonstrated to support the derivation of a myogenic cell lines capable of extended passage. Non-stochastic methods for generating skeletal muscle cell lines from fish, poultry and livestock species have not been reported. To date, animal biomass has not been commercially manufactured by ex vivo cultivation for dietary consumption. Various food product prototypes manufactured from animal biomass have been documented. However, the cell stocks used to manufacture these prototypes are limited to the expansion scale permitted by the cell stock's genetic program, which in normal somatic cells used, entails replicative senescence.

One approach to circumvent replicative senescence employs directing the ontological lineage commitment of previously established pluripotent or non-myogenic cell lines with an indefinite renewal capacity to the myogenic lineage. See U.S. patent application Ser. No. 15/134,252, which is herein incorporated by reference. By contrast, an alternate approach herein described is a reciprocal process that indefinitely extends the replicative capacity of lineage-committed muscle cells to generate myogenic cell lines for scalable manufacturing of biomass for dietary consumption and industrial bioprocess applications.

### SUMMARY OF INVENTION

The invention is a product and process using genetic amendments for extending the replicative capacity of metazoan somatic cells by abrogating retinoblastoma protein inhibition of the cell cycle during proliferation, but not during differentiation; while maintaining telomerase activity, to lessen or abolish replicative senescence characteristics of normal, unmodified somatic cells and derive clonal cell lines for scalable applications in the industrial production of metazoan cell biomass.

In one example of the present invention, where the application is biomass manufactured for dietary consumption, the species identity of the cells is Gallus gallus and the cell lineage is skeletal muscle. In one embodiment, the genetic amendments of this invention constitute direct inactivation of proteins representing inhibitors of CDK4 “INK4” CM homologs p15 and p16 (which are inhibitors of Cyclin-dependent kinase 4 “CDK4” (hence their name INhibitors of CDK4)). Inactivation of p15 and p16 is achieved by mutating the conserved nucleotide sequences encoding INK4 proteins encoded by the INK4B-ARF-INK4A locus to extend the proliferative capacity of the targeted cell populations.

Specifically, the first exon of the CDKN2B gene is targeted to disrupt the p15 protein within primary cell populations isolated from Gallus gallus skeletal muscle. An insertion or deletion mutation (INDEL) using guide RNAs targeting the first exon is created using clustered regularly-interspaced short palindromic repeats-Cas9 (CRISPR/Cas9). This demonstrates that disruption of CDKN2B locus alone is sufficient to increase the proliferative capacity in modified cell populations relative to their unaltered parental populations.

Nonetheless, when these amendments are combined with ancillary telomerase activity from a genetic construct directing expression of a telomerase protein homolog (e.g., from an ectopic TERT gene), the replicative capacity of the modified cell populations increases indefinitely. Indicators for proliferation and senescence are scored with respect to the unaltered primary cell populations to validate the approach.

Cells of a female Gallus gallus species karyotype were chosen to model this process for several reasons. First, the female karyotype of Gallus gallus constitutes the heterogametic sex. Since the CDKN2B allele is located on a sex chromosome of Gallus gallus, targeting only one allele renders this genome nullizygotic in female animals. Second, the Gallus gallus genome lacks an ortholog of the mammalian CDKN2A gene encoding the p16 protein. Thus, the inactivation of only one INK4 encoding-gene is necessary to ablate all p15/p16 activity in this model.

### BRIEF DESCRIPTION OF THE DRAWINGS

FIG. 1 shows insertion of a transgenic DNA sequence containing a gene (example shown: Serum Albumin), preceded by, and under the regulation of, an active regulatory region. (Example shown: Desmin Promoter).

FIG. 2 shows insertion of a transgenic DNA sequence containing an active regulatory region (example shown: Desmin Promoter) for regulation of expression from a target gene (Example shown: Serum Albumin) endogenous to the genome.

FIG. 3 shows genetic amendment of an endogenous regulatory region (example shown: Serum Albumin Promoter) DNA sequence by INDEL or point mutation, for the activation of a regulatory region (example shown: Serum Albumin Gene).

FIG. 4 shows insertion of a transgenic DNA sequence representing a target gene (example shown: Serum Albumin Gene) for expression under the regulation of an endogenous, 5′ regulatory region with tissue-specific activity (example shown: Myostatin Promoter), and parallel disruption of the regulatory region's activation of the endogenous gene 3′ to the inserted DNA sequence (example shown: Myostatin Gene).

FIG. 5 shows epigenetic or transcription program inductive amendment for targeted activation of gene expression from the promoter region of a targeted gene (example shown: Serum Albumin Promoter & Gene), alone or in combination with, activation of gene expression from an enhancer outside of the regulatory region by a genetic sequence-targeted activation tether. For exemplary purposes, modalities representing CRISPRa (i.e. CRISPR activation) are shown.

FIG. 6 shows epigenetic- or transcription program-suppressive amendment for targeted repression of gene expression from the regulatory region of a target gene, or directly from the coding sequence of the target gene, by a DNA-sequence targeted repression tether (example shown: Serum Albumin Promoter and Gene). For exemplary purposes, modalities representing CRISPRi (i.e. CRISPR interference) are shown.

FIG. 7 shows genetic amendment of an endogenous regulatory region (example shown: Myostatin Promoter) or the target gene coding sequence, such as the start codon, by an INDEL or point mutation, to silence or disrupt expression of the endogenous target gene (example shown: Myostatin Gene).

FIG. 8 shows insertion of a transgenic DNA sequence containing an shRNA coding region (example shown: Myostatin shRNA), preceded by, and under the regulation of, an active regulatory region (example shown: Desmin promoter).

FIG. 9 shows p16 and p15 amino acid alignments: a. Conservation among predicted amino acid sequence homology within representative mammalian murine (Mus musculus; SEQ ID NO 17), bovine (Bos taurus; SEQ ID NO 18), and porcine (Sus scrofa; SEQ ID NO 19) p16 orthologs (Panel A); b. Conservation among predicted amino acid sequence homology within representative metazoan gallinacean (Gallus gallus; SEQ ID NO 20), murine (Mus musculus; SEQ ID NO 21), bovine (Bos taurus; SEQ ID NO 23), porcine (Sus scrofa; SEQ ID NO 22), salmonid (Oncorhynchus mykiss; SEQ ID NO 24) and cichlid (Oreochromis niloticus; SEQ ID NO 25) p15 orthologs (Panel B).

### DETAILED DESCRIPTION

The genetic amendments of this invention decouple retinoblastoma protein inhibition of cell division cycle advancement during replicative senescence. This is accomplished by abrogating CM-mediated stabilization of the retinoblastoma protein, as shown in exemplary embodiments. We disclose three exemplary embodiments: (I) Inactivation of the p15 protein by disruption of the CDKN2B gene through targeted mutation of the coding sequence; (II) Inactivation of the p16 protein by disruption of the CDKN2A gene through targeted mutation of the coding sequence; and (III) Abrogation of endogenous CKI-mediated stabilization of retinoblastoma protein inhibition of cell division cycle progression by overexpression of a cyclin-dependent kinase homolog. These amendments, alone, or in combination, with ancillary overexpression of telomerase directed by an ectopic genetic construct, extend the replicative capacity of the metazoan cells amended.

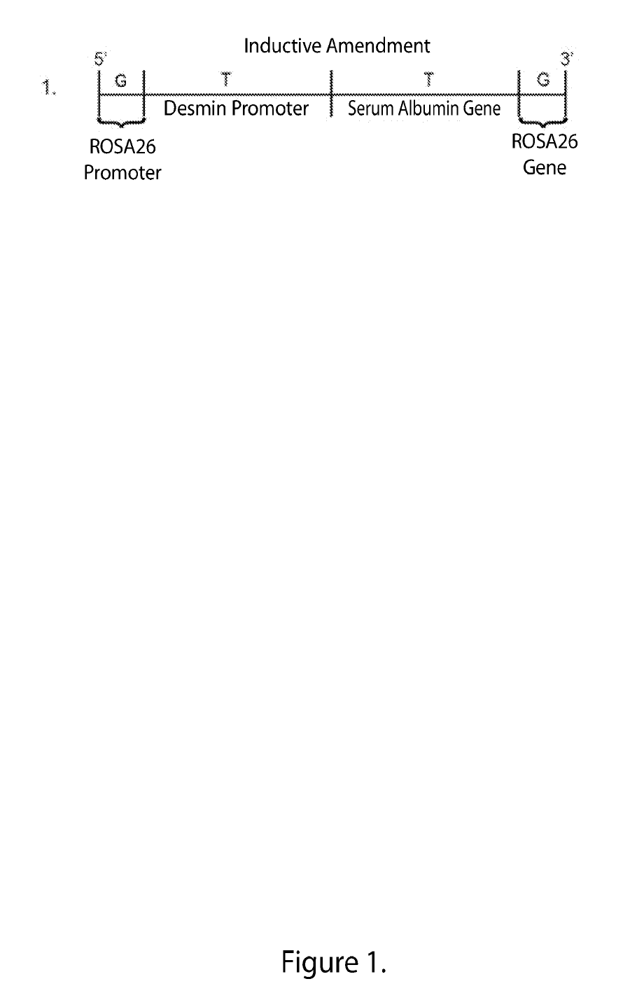

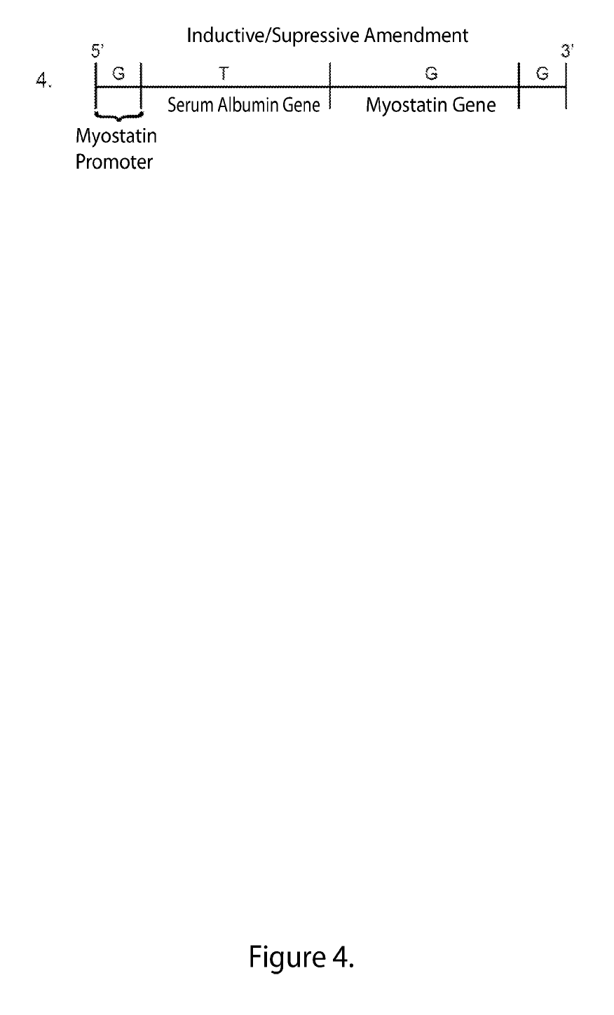

Illustrated in FIG. 1-5 are five exemplary models of inductive amendment for targeted transcriptional activation of a gene and four exemplary models of suppressive amendment (FIG. 4, 6-8) for targeted suppression of gene products. In FIG. 1-8, G refers to the native genomic DNA sequence and T refers to the foreign transgenic DNA sequence. Note that FIG. 4 may represent an inductive amendment to the transgene introduced and a suppressive amendment to the endogenous gene separated from its native promoter. For the purpose of this example, the wild-type host cell tissue lineage is Myostatin⁺/Desmin⁺ skeletal muscle. For illustrative purposes, arrows indicate exemplary regions of genetic amendment within the DNA sequence of the promoter and/or gene by INDEL or point mutation. ROSA26 indicates an exemplary, transcriptionally active amendment locus. Amendments may include, for example, alterations to the endogenous or unmodified genetic, epigenetic or transcription program.

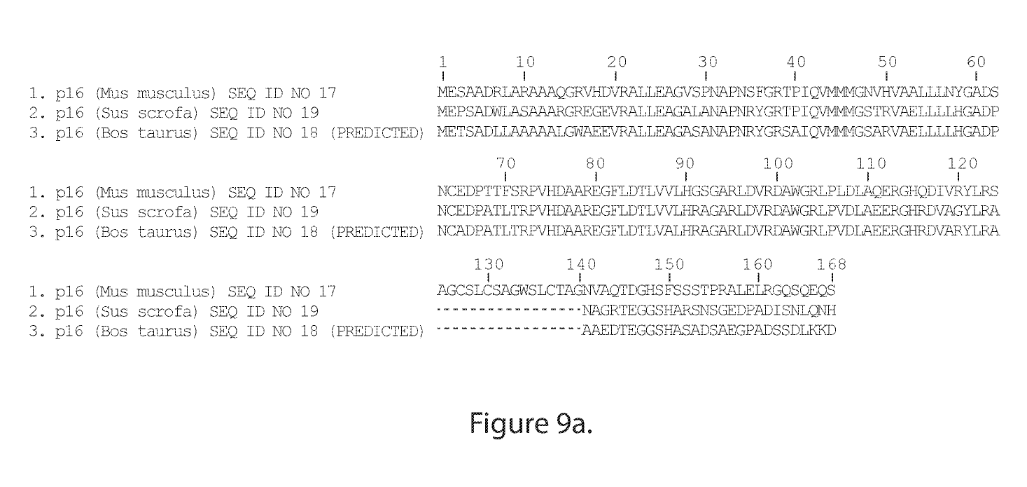

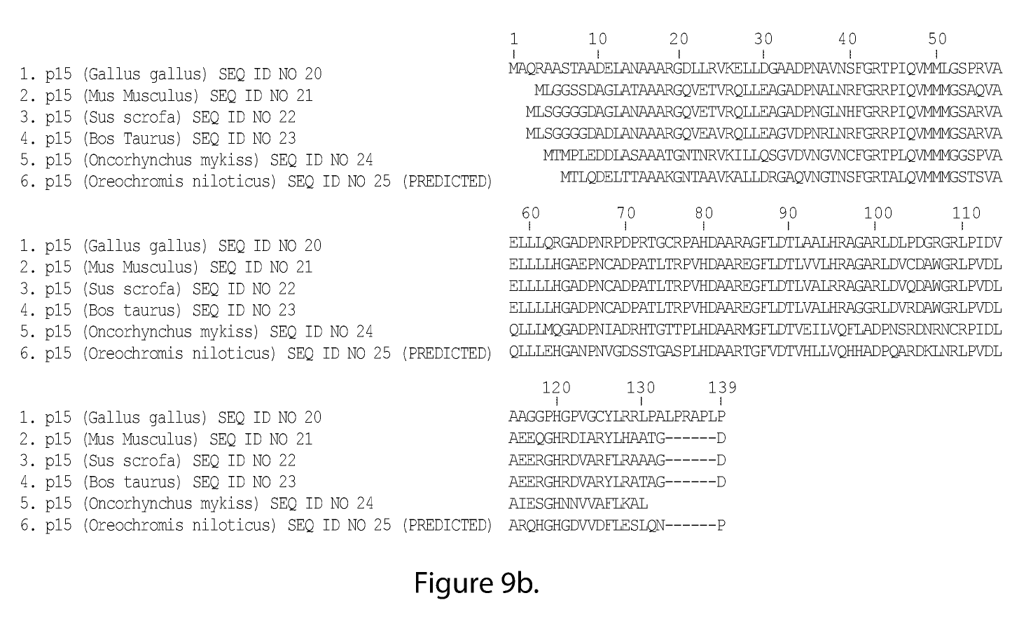

Among species commonly accepted as classified as livestock, the predicted amino acid sequences of the protein p16 are largely conserved with 82% pairwise identity between Bos taurus (SEQ ID NO 18) and Sus scrofa (SEQ ID NO 19). Likewise, among species classified as livestock and seafood, the amino acid sequences of the protein p15 are conserved as follows: 92% pairwise identity between Bos taurus (SEQ ID NO 23) and Sus scrofa (SEQ ID NO 22), 60% between Oncorhynchus mykiss (SEQ ID NO 24) and Oreochromis niloticus (SEQ ID NO 25). Further, comparing all livestock, poultry and seafood at the p15 locus (shown in FIG. 9 panel B) the genomic alignment reveals that there is 58% pairwise identity (Gallus gallus, SEQ ID NO 20; Mus musculus, SEQ ID NO 21; Bos taurus, SEQ ID NO 23; Sus scrofa, SEQ ID NO 22; Oncorhynchus mykiss, SEQ ID NO 24; and Oreochromis niloticus, SEQ ID NO 25).

FIG. 9 demonstrates p16 and p15 amino acid alignments: a. Conservation among amino acid sequence homology within representative mammalian murine (Mus musculus; SEQ ID NO 17), bovine (Bos taurus; SEQ ID NO 18), and porcine (Sus scrofa; SEQ ID NO 19) p16 orthologs (Panel A); b. Conservation among amino acid sequence homology within representative metazoan gallinacean (Gallus gallus; SEQ ID NO 20), murine (Mus musculus; SEQ ID NO 21), bovine (Bos taurus; SEQ ID NO 23), porcine (Sus scrofa; SEQ ID NO 22), salmonid (Oncorhynchus mykiss; SEQ ID NO 24) and cichlid (Oreochromis niloticus; SEQ ID NO 25) p15 orthologs (Panel B). Darkened areas indicate degree of conservation (i.e., identity and similarity): black shading indicates 100% similarity, grey indicates 60-80% similarity and white represents less than 60% similarity based on the Blosum 62 score matrix (images and scoring produced in Geneious R10 (http://www.geneious.com, Kearse et al., 2012).

Together, these orthologs demonstrate conserved amino acid sequence homology of p15 and p16 orthologs targeted by embodiments of this invention among species classified as livestock, poultry and seafood. Note that annotated fish and avian genomes lack a p16 ortholog. Therefore, it is predicted that the integrity of p15-mediated function across avian, mammalian and fish species is conserved and the integrity of p16-mediated function across mammalian species is conserved as a platform for targeted application in this invention.

### First Exemplary Embodiment: Delayed Replicative Senescence Through Genetic Amendment of the CDKN2B Locus and Ectopic Expression of TERT

### (A) Delayed Replicative Senescence Through Genetic Amendment of the CDKN2B Locus

In sum, this first exemplary embodiment disrupts the CDKN2B locus encoding the p15 protein within primary myoblast cell populations isolated from Gallus gallus skeletal muscle using CRISPR/Cas9 to generate an INDEL targeted to exon #1 of CDKN2B (NCBI Accession Number: NM_204433.1) using guide RNAs (gRNAs) (SEQ ID NO 1-5). Although disrupting the CDKN2B locus alone provides benefits, when done in combination with ancillary telomerase activity from a genetic construct (SEQ ID NO 6) directing expression of a telomerase protein homolog from a TERT gene (NCBI Accession Number: NM_001031007.1; NCBI Gene ID: 420972), the replicative capacity of modified cell populations increases indefinitely. Targeted inactivation of CDKN2B is a novel approach.

In general terms, the first embodiment extends the native replication capacity of metazoan somatic cells by ex vivo cultivation in a five step process: (1) decoupling retinoblastoma protein inhibition of cell division cycle advancement in replicative senescence by inactivating a p15 protein to abolish retinoblastoma protein inhibition of the cell cycle during proliferation, but not during differentiation; (2) maintaining telomerase activity by transducing the metazoan somatic cells with a genetic construct directing expression of a telomerase protein homolog from a functional TERT gene (“ectopic expression of TERT”) to lessen replicative senescence characteristic of normal, unmodified metazoan somatic cells; (3) maintaining a bank of cells having the CDKN2B gene mutation and ectopic expression of TERT (i.e., the “master cell bank”); (4) cultivating cells from the master cell bank in an ex vivo milieu; and (5) harvesting the cultivated cell biomass for dietary consumption.

More specifically and in one embodiment, the first exon of the CDKN2B gene (NCBI Accession Number: NM_204433.1) encoding the p15 protein is targeted for genetic amendment using gRNA-targeted CRISPR/Cas9 (gRNA SEQ ID NO 1-5) to insert a mutation within the first exon of the CDKN2B gene within the genome of a Gallus gallus cell population. For a more detailed discussion of the methods used for these examples see below in the Materials and Methods sections A, B, D, E, and F.

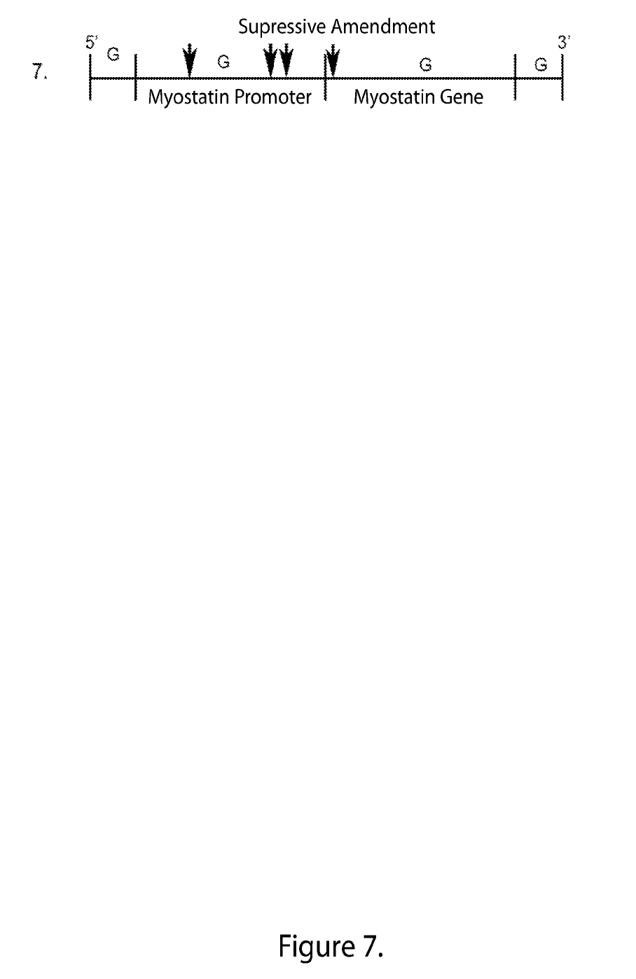

Exemplary embodiment I employs the model illustrated in FIG. 7 for disrupting the function of the endogenous p15 protein. Specifically, the model shown in FIG. 7 may be adapted for use in this invention where endogenous gene expression is disrupted by mutation of the start codon or a regulatory region sequence enabling transcriptional activation. Alternatively, endogenous gene function may be disrupted by introducing a frameshift mutation after the start codon. For instance, as applied to this invention, the model shown in FIG. 7 may represent the repression of p15 function by an INDEL mutation introduced into the 5-prime (5′) coding sequence of the CDKN2B gene using CRISPR/Cas9-targeted nuclease activity.

### (B) Expression of an Ectopic TERT Gene

Cells modified as described in Embodiment I-A may also be modified with a genetic construct directing expression of a telomerase protein homolog from a TERT gene (NCBI Accession Number: NM_001031007.1; NCBI Gene ID: 420972). This is followed by selection of cells from this population transduced with a genetic construct (SEQ ID NO 6) directing expression of a telomerase protein homolog from a TERT gene for cells featuring genetic amendment by the genetic construct directing expression of a telomerase protein homolog.

Exemplary embodiment I makes use of the model illustrated in FIG. 1 for expression of an ectopic TERT gene. Specifically, the model shown in FIG. 1 may be adapted for use in this invention where ectopic genes are expressed under the regulation of a foreign promoter. For instance, as applied to this invention, the model shown in FIG. 1 may represent expression of an ectopic TERT gene from an introduced CAG promoter.

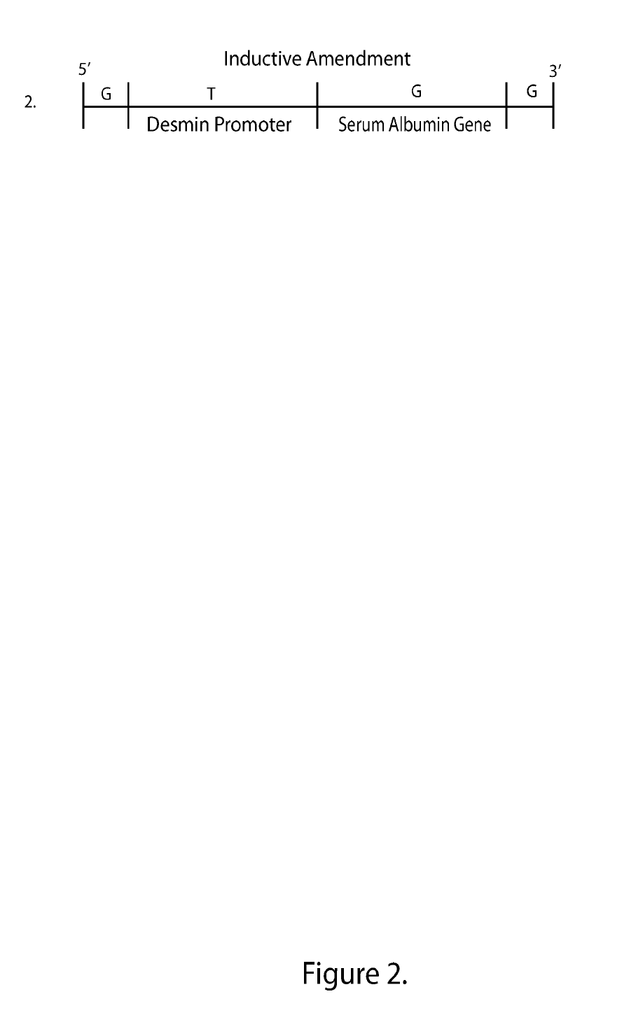

The model shown in FIG. 2 may be adapted for use in this invention where endogenous genes are expressed under the regulation of a foreign promoter. For instance, as applied to this innovation, the model shown in FIG. 2 may also represent the expression of an endogenous TERT gene from an introduced CAG promoter.

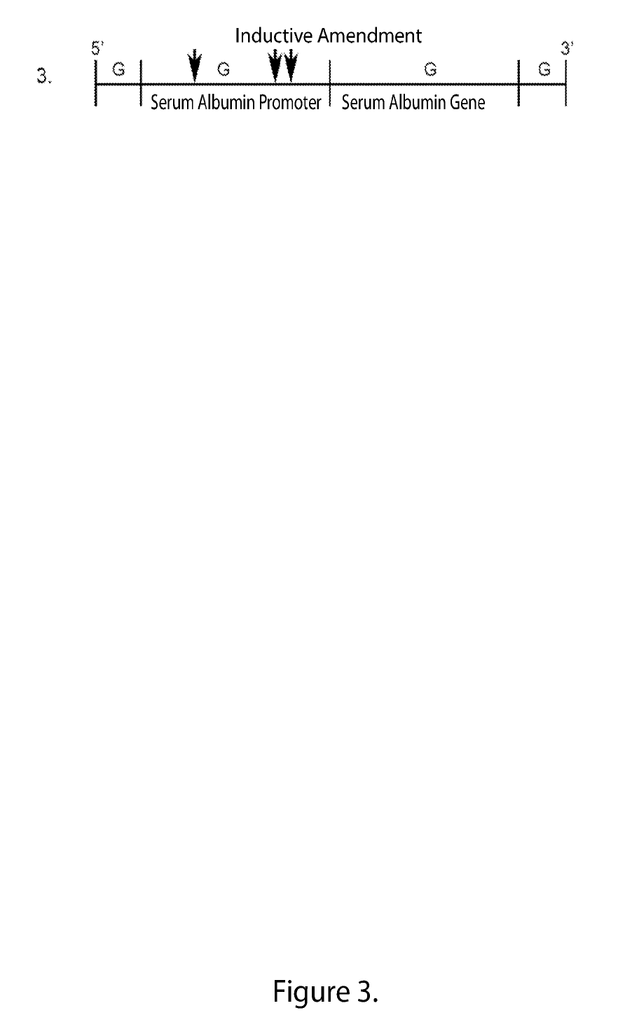

The model shown in FIG. 3 may be adapted for use in this invention where gene expression is altered by targeted mutation of its regulatory sequence. For instance, as applied to this invention, the model shown in FIG. 3 may represent the expression of an endogenous TERT gene from an endogenous TERT promoter wherein repressor binding sites within the TERT promoter have been disrupted by mutation.

The model shown in FIG. 4 may be adapted for use in this invention where the expression of a foreign gene is expressed under the regulation of an endogenous promoter inserted at the 3-prime (3′) end of the gene. For instance, as applied to this invention, the model shown in FIG. 4 may represent the expression of a foreign TERT gene from and endogenous (3-actin promoter.

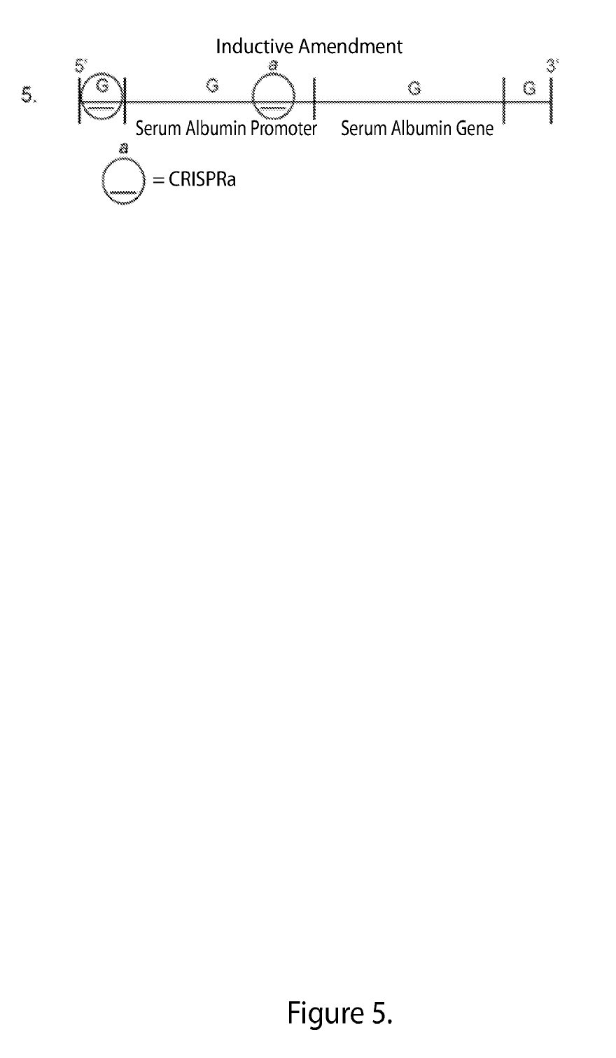

The model shown in FIG. 5 may be adapted for use in this invention where the expression of an endogenous gene is transcriptionally activated from the promoter by a genetic sequence-targeted activation tethering. For instance, as applied to this invention, the model shown in FIG. 5 may represent the induction of an endogenous TERT gene by CRISPRa targeted to the TERT promoter. Exemplary CRISPRa modalities include, but are not limited to, activation mediated by a nuclease-deficient Cas9 protein targeted to the regulatory region of a target gene by a single gRNA. Outside of the scope of CRISPRa, modalities for this inductive amendment mechanism encompass genetic sequence-targeted activation tethering including, but not limited to, nuclease-deficient Zinc Finger Nucleases (ZFNs) and nuclease-deficient Transcription Activator-Like Effector Nucleases (TALENs).

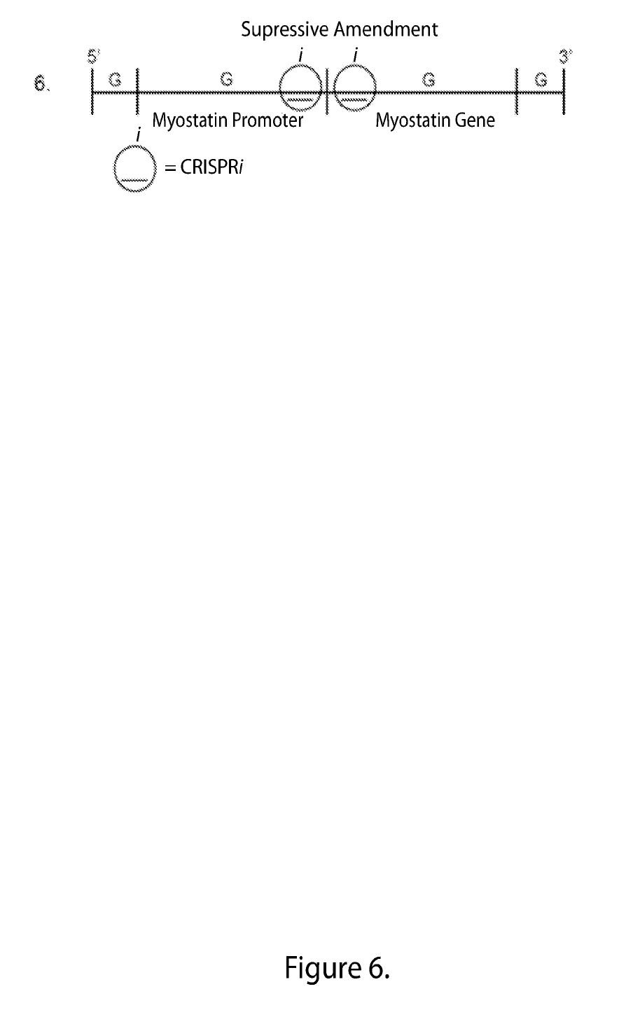

The model shown in FIG. 6 may be adapted for use in this invention where the expression of an endogenous gene is transcriptionally repressed from the promoter by genetic sequence-targeted repression tethering. For instance, as applied to this invention, the model shown in FIG. 6 may represent the repression of the endogenous CDKN2B gene by CRISPRi targeted to the CDKN2B promoter. Exemplary CRISPRi repression modalities include, but are not limited to, repression mediated by nuclease-deficient Cas9 protein targeted to a regulatory region or gene by a single gRNA. Outside of the scope of CRISPRi, modalities for this suppressive amendment mechanism encompass DNA sequence-targeted repression tethering including, but not limited to, nuclease-deficient ZFNs and nuclease-deficient TALENs.

For a more detailed discussion of the methods used in these examples see the Materials and Methods below in paragraphs C, G, H and I; noting the manufacturing process can be accomplished in this exemplary embodiment using the same manufacturing process described above in section I-B.

### (C) Manufacturing Biomass for Dietary Consumption

In one embodiment, manufacturing biomass for dietary consumption comprises four steps: (1) Expanding selected cell populations harboring genetic amendment to the CDKN2B locus and/or ectopic expression of TERT; (2) Cryopreserving and storing expanded cell populations in a master cell bank stock inventory; (3) Seeding and cultivating cells from master cell bank stock inventory in an ex vivo milieu; and (4) Harvesting cultivated cell biomass for dietary consumption.

Step 1 is expanding selected cell populations harboring genetic amendment to the CDKN2B locus. Selected cell populations harboring the genetic amendment are seeded onto a substrate consisting of gelatin-coated tissue-culture treated plastic, in a standard growth medium containing 10% animal serum, such as, but not limited to, bovine serum plus basal medium at a density of 7.5×10³ cells/cm² and cultured at 37° C. under 5% carbon dioxide, 5% oxygen atmospheric conditions. As cultures approach 80% confluence, cells are enzymatically dissociated and the expanded quantity of cells is seeded at 7.5×10³ cells/cm². This process is repeated until the total number of cells harvested following dissociation exceeds 1.0×10⁸ cells.

Step 2 is cryopreserving and storing the expanded cell populations in a master cell bank stock inventory. Cells are harvested in quantities equal to or exceeding 1.0×10⁸. Following expansion, selected cells are pelleted by centrifugation for 5 minutes at 300×g. The cell pellet is suspended in a standard cryopreservation medium at 2.5×10⁶ cells/mL and aliquoted at 1.0 mL per cryovial. Cryovials are cooled to −80° C. at 1° C./minute using a controlled cooling container and transferred to a dewar containing liquid nitrogen for long-term storage. As cells stocks are depleted from this bank, remaining vials of cells are expanded as described in Embodiment I-B-1 and cryopreserved as described in Embodiment I-B-2 to replenish and expand the master cell bank inventory.

Step 3 is seeding and cultivating cells from a master cell bank in an ex vivo milieu: In accordance with the cultivation scale desired, one or more vials from the master cell bank is rapidly thawed to room temperature. The cryopreservation medium is removed from the cells by a 5 minute, 300×g centrifugation step. Cells are suspended in standard growth medium and seeded onto a gelatin-coated cultivation substrate in standard growth medium and cultivated as outlined in embodiment I-B-1, except that, on the final passage prior to harvest, the cells are permitted to proliferate to equal to, or greater than, 100% confluence on the cell culture substrate. The cultivation scale for biomass harvest is outlined according to Table 1, where the estimated average cell mass is 2.0×10 grams, and the estimated average cell doubling time is 24 hours (h).

TABLE 1

| # hours | | 1 vial | 2 vials | 3 vials | 4 vials | 5 vials | 6 vials | 7 vials | 8 vials | 9 vials | 10 vials |
|---|---|---|---|---|---|---|---|---|---|---|---|
| 0 | h | 0.005 | 0.01 | 0.015 | 0.02 | 0.025 | 0.03 | 0.035 | 0.04 | 0.045 | 0.05 |
| 24 | h | 0.01 | 0.02 | 0.03 | 0.04 | 0.05 | 0.06 | 0.07 | 0.08 | 0.09 | 0.1 |
| 48 | h | 0.02 | 0.04 | 0.06 | 0.08 | 0.1 | 0.12 | 0.14 | 0.16 | 0.18 | 0.2 |
| 72 | h | 0.04 | 0.08 | 0.12 | 0.16 | 0.2 | 0.24 | 0.28 | 0.32 | 0.36 | 0.4 |
| 96 | h | 0.08 | 0.16 | 0.24 | 0.32 | 0.4 | 0.48 | 0.56 | 0.64 | 0.72 | 0.8 |
| 120 | h | 0.16 | 0.32 | 0.48 | 0.64 | 0.8 | 0.96 | 1.12 | 1.28 | 1.44 | 1.6 |
| 144 | h | 0.32 | 0.64 | 0.96 | 1.28 | 1.6 | 1.92 | 2.24 | 2.56 | 2.88 | 3.2 |
| 168 | h | 0.64 | 1.28 | 1.92 | 2.56 | 3.2 | 3.84 | 4.48 | 5.12 | 5.76 | 6.4 |
| 192 | h | 1.28 | 2.56 | 3.84 | 5.12 | 6.4 | 7.68 | 8.96 | 10.24 | 11.52 | 12.8 |
| 216 | h | 2.56 | 5.12 | 7.68 | 10.24 | 12.8 | 15.36 | 17.92 | 20.48 | 23.04 | 25.6 |
| 240 | h | 5.12 | 10.24 | 15.36 | 20.48 | 25.6 | 30.72 | 35.84 | 40.96 | 46.08 | 51.2 |
| 264 | h | 10.24 | 20.48 | 30.72 | 40.96 | 51.2 | 61.44 | 71.68 | 81.92 | 92.16 | 102.4 |
| 288 | h | 20.48 | 40.96 | 61.44 | 81.92 | 102.4 | 122.88 | 143.36 | 163.84 | 184.32 | 204.8 |
| 312 | h | 40.96 | 81.92 | 122.88 | 163.84 | 204.8 | 245.76 | 286.72 | 327.68 | 368.64 | 409.6 |
| 336 | h | 81.92 | 163.84 | 245.76 | 327.68 | 409.6 | 491.52 | 573.44 | 655.36 | 737.28 | 819.2 |

Table 1 shows Biomass Production Scale Cultivation Yield Estimates. Masses are shown in grams. 1 vial is equivalent to ˜2.5×10⁶ cells.

Step 4 is harvesting cultivated cell biomass for dietary consumption. After the cells have proliferated to confluence, the culture medium is removed and the adherent cell cultures are rinsed with phosphate buffered saline. Next, the confluent biomass of adherent cells is mechanically dissociated from the substrate by means of a scraping device. The dissociated biomass is collected into centrifuge tubes, pelleted at 400×g for 5 minutes to remove excess liquid and processed for food product formulation.

### Second Exemplary Embodiment: Delayed Replicative Senescence Through Genetic Amendment of the CDKN2A Locus and Ectopic Expression of TERT

In sum, the second exemplary embodiment disrupts the CDKN2A locus encoding the p16 protein within a metazoan somatic cell population that is a primary myoblast cell population isolated from Bos taurus skeletal muscle using CRISPR/Cas9 to create an INDEL mutation targeted to the first exon of the CDKN2A sequence encoding p16, using gRNAs (SEQ ID NO 8-10). The Bos taurus gene CDKN2A has two predicted splice variants (NCBI Accession Numbers: XM_010807759.2, XM_010807758.1) wherein the first exon encoding p16 is exon #2 of CDKN2A. Although disrupting the CDKN2A locus alone provides replicative benefits, when used in combination with ancillary telomerase activity from a synthetic genetic construct (SEQ ID NO 11) directing expression of a telomerase protein homolog from a TERT gene in the same cell population (NCBI Accession Number: NM_001046242.1; NCBI Gene ID 518884), the synergistic action of both amendments can increase the replicative capacity of the modified cell population further than either amendment alone. The Bos taurus species genome features a CDKN2A gene with a predicted transcript encoding p16 (NCBI Accession Number: XM_010807759.2). In other words, the genetic amendment is a mutation of a conserved nucleotide sequences in exon two of the CDKN2A gene (i.e., exon one of the gene sequence encoding p16) and guide RNAs targeting exon two of the CDKN2A gene (i.e., exon one of the gene sequence encoding p16) and is created using CRISPR/Cas9. Disrupting the CDKN2A locus within the predicted p16 coding sequence is a novel method in this application. For a more detailed discussion of the methods used for these examples see Materials and Methods sections A, B, C, D, E, F, G, H and I.

In all other regards the second exemplary embodiment follows the method outlined in the first exemplary embodiment to manufacture biomass for dietary consumption in an ex vivo cultivation process, which comprises: (1) decoupling retinoblastoma protein inhibition of cell division cycle advancement in replicative senescence by inactivating a p15 protein to abolish retinoblastoma protein inhibition of the cell cycle during proliferation, but not during differentiation; (2) maintaining telomerase activity by transducing the metazoan somatic cells with a genetic construct directing expression of a telomerase protein homolog from a functional TERT gene (“ectopic expression of TERT”) to lessen replicative senescence characteristic of normal, unmodified metazoan somatic cells; (3) maintaining a bank of cells having the CDKN2A locus mutation and ectopic expression of TERT (“master cell bank”); (4) cultivating cells from the master cell bank in an ex vivo milieu; and (5) harvesting the cultivated cell biomass for dietary consumption.

Exemplary Embodiment II employs the model illustrated in FIG. 7 for disrupting the function of the endogenous p16 protein; and Exemplary Embodiment II employs the model illustrated in FIG. 1 for expression of an ectopic TERT gene.

### Third Exemplary Embodiment: Delayed Replicative Senescence Through Expression of Ectopic Cyclin-Dependent Kinase and Ectopic TERT

In general terms, the third exemplary embodiment abrogates cyclin-dependent kinase inhibitor-mediated stabilization of retinoblastoma protein inhibition of the cell division cycle during replicative senescence by ectopic overexpression of cyclin-dependent kinase homologs; specifically, modification of cells with a genetic construct directing ectopic expression of a CDK4 protein homolog, from a CDK4 gene (NCBI Gene ID: 510618). Additional benefit can be achieved by adding ancillary telomerase activity from a genetic construct directing overexpression of a telomerase protein homolog from a TERT gene (NCBI Gene ID: 518884). Overexpression of telomerase protein homolog increases the replicative capacity of modified cell populations relative to the unmodified parental populations.

More specifically and in one embodiment, Gallus gallus was chosen to model modification of poultry skeletal muscle cells using a genetic construct directing ectopic expression of a cyclin-dependent kinase protein homolog from a cyclin-dependent kinase gene; maintaining a master cell bank stock inventory of cells harboring the genetic construct directing ectopic expression of a cyclin-dependent kinase protein homolog from a cyclin-dependent kinase gene. Transduction of said metazoan cell population is with a genetic construct (SEQ ID NO 12) directing expression of a CDK4 protein homolog from a Bos taurus CDK4 gene (NCBI Accession Number: NM_001037594.2; NCBI Gene ID: 510618). For a more detailed discussion of the methods used for these examples see the Materials and Methods sections C, G, H, and I.

Manufacturing biomass for dietary consumption and modification of cells from the metazoan cell population with a genetic construct (SEQ ID NO 11) directing expression of a telomerase protein homolog from a TERT gene follows the method described above in the first exemplary embodiment.

Exemplary embodiment III employs the model illustrated in FIG. 1 for expression of an ectopic CDK4 gene. Exemplary Embodiment III employs the model illustrated in FIG. 1 for expression of an ectopic TERT gene.

### <Materials and Methods>

### (A) Target Locus Sequencing

Genomic DNA is extracted from primary cell isolates with the E.Z.N.A. Tissue DNA Extraction kit (Omega Bio-tek) for each species. Genomic DNA is PCR amplified using primers designed to amplify endogenous CDKN2B from Gallus gallus and endogenous CDKN2A from Bos Taurus.

In Gallus gallus, the putative promoter region and CDKN2B are PCR amplified using primers SEQ ID NO 26-31; see Table 2 below providing details on primer targets. In Bos taurus, the putative promoter region and CDKN2A are PCR amplified using primers SEQ ID NO 32-43; see Table 2 below for details on primer targets.

TABLE 2

| SEQ ID NO | DNA Sequence | Details |
|---|---|---|
| 1 | TCACCCGCAG CAGATCGCCG | gRNA targeting G.gallus CDKN2B |
| | CGG | exon 1 |
| 2 | CGGGTGAAGG | gRNA targeting G.gallus CDKN2B |
| | AGCTACTGGA CGG | exon 1 |
| 3 | GCACCACGCC TGCTGCTCCG | gRNA targeting G.gallus CDKN2B |
| | GGG | exon 1 |
| 4 | GCTGGGCTCC CCTCGCGGGT | gRNA targeting G.gallus CDKN2B |
| | CGG | exon 1 |
| 5 | CCTCGTGTCT GTGGGCAGCG | gRNA targeting G.gallus CDKN2B |
| | GGG | exon 1 |
| 8 | GGCGGCCAAC | gRNA targeting bovine CDKN2A exon |
| | AAGTCGGCCG AGG | 2 |
| 9 | GCCAACGCGC CGAACCGTTA | gRNA targeting bovine CDKN2A exon |
| | CGG | 2 |
| 10 | CCTCGGGTGC AAAGACTCCG | gRNA targeting bovine CDKN2A exon |
| | CGG | 2 |
| 26 | CTCTCCGTCC TCCCTACCTG | Primer for G.gallus CDKN2B exon 1 |
| 27 | GTACCAACTG | Primer for G.gallus CDKN2B exon 1 |
| | CGGGGAGAAA | |
| 28 | GGACGCCGGT CAATGAATCA | Primer for G.gallus CDKN2B exon 2 |
| 29 | CAGGTGATGA TGCTGGGCAG | Primer for G.gallus CDKN2B exon 2 |
| 30 | TTTCTCCCCG CAGTTGGTAC | Primer for G.gallus CDKN2B promoter |
| 31 | CTGCAAGACC CAAGACGTCT | Primer for G.gallus CDKN2B promoter |
| 32 | GCCTAGTCCC ACACCCTTTC | Primer for bovine CDKN2A promoter |
| 33 | CATTTAAGCC TGGCCCCTGA | Primer for bovine CDKN2A promoter |
| 34 | TGTCCGACTC TTTGCCATCC | Primer for bovine CDKN2A exon 4 |
| 35 | GACCCTGGAT AAGGCGTCAG | Primer for bovine CDKN2A exon 4 |
| 36 | AGTGAATGCT CTGGGAAGCG | Primer for bovine CDKN2A exon 3 |
| 37 | GATTGTCAGC GCATCTGCAG | Primer for bovine CDKN2A exon 3 |
| 38 | TAGAGATCTG AACCCCACGC | Primer for bovine CDKN2A exon 2 |
| 39 | CTCTGATGGG | Primer for bovine CDKN2A exon 2 |
| | AGTGGGGAGA | |
| 40 | AGGCCTTTCC TACCTGGTCT | Primer for bovine CDKN2A exon 1 |
| 41 | TAATTCCGCT GGTTTCCCAA | Primer for bovine CDKN2A exon 1 |
| 42 | AAACTGCTGC GACATCTGGA | Primer for bovine CDKN2A promoter |
| 43 | ACGGTCCCTC TTCTCTCTCC | Primer for bovine CDKN2A promoter |

Table 2 shows details of gRNA and primer sequences.

PCR products were compared to the in silico predicted sequence size using gel electrophoresis (1% agarose run at 10 W/cm). To determine the genomic sequence of CDKN2A and CDKN2B, the respective PCR products are purified using a commercial magnetic bead kit and Sanger sequenced. Sequencing primers are the same as those used to amplify the initial PCR product. All PCR primers are designed using a modified version of Primer3 2.3.7 (Untergasser et al., 2012), available in Geneious R10 (http://www.geneious.com, Kearse et al., 2012), using the reference sequence for Gallus gallus CDKN2B (NCBI Accession Number NM_204433.1) and Bos taurus CDKN2A (NCBI Accession Number: XM_010807759.2). The reference chromosome assembly for each species (NCBI Accession Number: AC_000165 for Bos taurus, NCBI Accession Number: NC_006127 for Gallus gallus) is used to design primers which amplify the putative promoter region for each gene

(B) gRNA Design

CRISPR/Cas9 is used to disrupt p16 of CDKN2A in the Bos taurus genome and p15 of CDKN2B in Gallus gallus genome. Suitable gRNAs for CRISPR/Cas9 are designed using the “Find CRISPR Sites” function in Geneious R10 (http://www.geneious.com, Kearse et al., 2012). Off-target effects are screened using the latest reference genome on NCBI for each species: Gallus gallus-5.0 (NCBI RefSeq Assembly Accession Number:GCF_000002315.4.) and Bos taurus v3.1.1 (NCBI RefSeq Assembly Accession Number: GCF_000003055.6). Only gRNAs with off target scores>90% are considered for synthesis. Guide RNA sequences selected for synthesis include but are not limited to SEQ ID NO 1-5 for Gallus gallus and SEQ ID NO 8-10 for Bos taurus. Separate pGS-gRNA vectors are constructed, amplified and purified for each selected gRNA by a third-party vendor. The pSpCas9 PX165 vector to be co-transfected with each gRNA plasmid is also sourced from a third-party vendor.

### (C) Synthetic Construct Design

The Cytomegalovirus, Chicken Beta-Actin, Rabbit Beta-Globulin (CAG) regulatory element (Alexopoulou, Couchman, and Whiteford 2008) is used to promote robust expression of all introduced synthetic constructs. The Gallus gallus TERT reference sequence (NCBI Accession Number: NM_001031007.1) was retrieved from NCBI. For each transcript variant (NM_001031007.1 and XM_015282334.1), the coding DNA sequence (CDS) regions are extracted and joined into a concatenated sequence. The CAG regulatory element is then joined to the 5′ end of the CDS for each transcript variant in Geneious R10 (http://www.geneious.com, Kearse et al., 2012). The full synthetic constructs SEQ ID NO 6 and SEQ ID NO 7 are assembled and cloned into a mammalian expression vector by a commercial vendor. The strategy described here is also used to create a bovine version of the synthetic TERT construct. The Bos taurus TERT construct (SEQ ID NO 11) is designed using the reference sequence (NCBI Accession Number: NM_001046242.1).

To drive strong expression of CDK4 in bovine cells another set of synthetic constructs was created using the method described above. The Bos taurus CDK4 reference sequence (NCBI Accession Number: NM_001037594.2) was retrieved from NCBI. For both the wild type and mutant sequences the CDS regions were extracted and joined into a concatenated sequence. The synthetic constructs SEQ ID NO 12 were assembled and cloned into a mammalian expression vector by a third-party vendor.

(D) CRISPR/Cas9 Cell Transfection for INDEL Mutagenesis of Gallus gallus CDKN2B or Bos Taurus CDKN2A

To perform targeted INDEL mutagenesis, gRNA sequences recognized by CRISPR/Cas9 proteins encoded by plasmids are synthesized, amplified, and purified for transfection. The plasmid DNA contains a standard vector backbone containing the necessary regulatory elements for amplification in prokaryotic systems. Each plasmid also contains the regulatory and coding regions necessary to express the gRNA or Cas9 protein transiently within each target cell population to incite INDEL mutation formation within the 5′ regulatory region, coding region, or both.

Delivery of the plasmid DNA constructs is mediated by a non-liposomal DNA complex-forming transfection reagent (Fugene HD, Promega) delivered directly to the primary cell isolations in suspension and during adhesion. gRNAs targeting the 5′ region of the Gallus gallus CDKN2B gene encoding the p15 or the 5′ region of the Bos taurus CDKN2A gene encoding p16 are designed for expression from the transfected plasmid. Transfection is performed by using modification of the Grunwald and Speer (2007) protocol for targeted alteration of primary human skeletal muscle myoblasts (Biochemica March 2007, pages 26-27). The prepared plasmid DNA is diluted to a working concentration of 1 μg/μL in sterile, deionized water. Each plasmid DNA stock is aliquoted in a 2:1 ratio of Cas9 plasmid DNA:gRNA plasmid for a total DNA amount of 2 μg and mixed well via trituration. The total DNA is diluted to a concentration of 1 μg/50 μL in sterile deionized water. Fugene HD is added to the DNA mixture at a ratio of 6:2, (Fugene HD reagent (in μL): total DNA (in μg)) and mixed well.

The mixture is incubated at room temperature for fifteen minutes to promote complexation with DNA. The incubated DNA mixture is diluted by adding an equal volume of sterile, deionized water. This entire reaction mixture is then added dropwise to cells in suspension. The cells and DNA reaction mixture is incubated at 37° C. under 5% CO₂, 5% O₂ atmospheric conditions for 16 hours.

Target cell populations are maintained in proliferation medium on tissue culture-treated plastic (6-well plates). The cells are maintained at a density of 4×10⁵ cells/ml of culture medium. Prior to transfection, the cell medium is removed aseptically, the cells washed in 1×PBS, and then exposed to 1× dissociation reagent (TrypLE-Gibco) at 37° C. for 3 minutes. The reaction is terminated with the addition of an equal volume of proliferation medium, followed by gentle trituration. The cells are then transferred to a 0.1% gelatin-coated well. At this point, the DNA transfection reaction complex is added, and the cells are incubated for 48 hours at 37° C. under 5% CO₂, 5% O₂ for 48 hours prior to further processing and evaluation.

### (E) Limiting Dilution of Transfected Cells Targeting CDKN2B or CDKN2A

Cell populations previously transfected using a CRISPR/Cas9 gRNA are selected by first isolating individual clonal populations of cells from the parental pool of cells subjected to one transfection cycle. Cells are selected using a limiting dilution method. Transfected cells are enzymatically-dissociated into a single cell suspension. A working stock of 2×10⁴ cells/mL proliferation medium is produced to a total volume of 500 μL. Each well in a tissue-culture treated 96 well plate is coated in 0.1% gelatin. With the exception of well A1, each well is pre-filled with 100 μL of proliferation medium. 4×10³ cells (200 uL) are transferred aseptically to the 0.1% gelatin-coated A1 well of a tissue-culture treated 96-well plate. One half of the volume (100 μL) of well A1 is immediately transferred to well B1 and mixed well. This dilution series (1:2) is repeated from well C1 through HE Upon receiving cells, 100 μL is removed and discarded from well HE 100 μL of cell-free proliferation medium is added to each well from A1-H1 and mixed well. Next, one half of the volume in well A1 is transferred to well A2 and mixed well. This process is repeated from A2 through A12. The dilution series is repeated again starting from well B1 and continued through B12. This entire process is repeated for rows C through H until the entire plate (all 96 wells) have received a dilution volume of cells. To complete the dilution series, each well in column 12 has one half (100 μL) removed and discarded. Each well in columns 2 through 12 have 100 μL of cell-free proliferation medium added to each to bring the final volume of each well on the plate to 200 μL. The entire plate is incubated at 37° C. under hypoxia until individual cell colonies emerge (greater than or equal to 10 cell doublings or 1,024 cells). Wells that appear to harbor cells originating from one single isolated cell are dissociated into single cells and passaged 1:1 into one well of a 0.1% gelatin coated 24 well plate and incubated at 37° C. under 5% CO₂, 5% O₂ atmospheric conditions. Once 90% confluent, cells are expanded to a 12-well then 6-well plate.

Cells isolated by limiting dilution and expanded to 90% confluence in a 6 well plate are then passaged 1:4 into a 6-well plate. 90% confluent wells are then processed for further evaluation. One well is dissociated into single cells, washed, and pelleted at 300×g for five minutes. Total cellular genomic DNA is isolated and quantified. Total genomic DNA is then subjected to PCR amplification of the CDKN2B or CDKN2A locus and run on a 1% agarose gel at 10 W/cm until resolved. These PCR products are then extracted from the agarose gel and submitted for Sanger sequencing. Sequence tracings are compared to the previously validated, wild-type, untreated parental cell population CDKN2B or CDKN2A genetic sequences.

### (F) Cell Proliferation and Senescence Assay (EdU Method)

Cell populations demonstrating INDEL mutation formation in the 5′ region of the Gallus gallus CDKN2B gene targeting p15 function, or the Bos taurus CDKN2A gene targeting p16 function, are then evaluated for functional and phenotypic characteristics via a standard EdU assay. Cell populations determined to harbor a correctly INDEL-disrupted gene and that are clonally pure are seeded at equal densities alongside wild-type cells that have received no previous CRISPR/Cas9 targeting and are of the same cell population doubling number as the INDEL-amended cells. Each cell type is allowed to incubate at 37° C. under 5% CO₂, 5% O₂ atmospheric conditions for 24 hours. Following this, a standard thymidine analog reagent (EdU) is added to the culture medium (Click-It Alexa 488 EdU kit, Thermo-Fisher) and the cells are incubated at 37° C. under 5% CO₂, 5% O₂ atmospheric conditions for 4 hours. Following incubation, the cells are washed, fixed, permeabilized, and probed using and anti-EdU Alexa 488-conjugated azide. Nuclear counter-staining is achieved using a standard DAPI nuclear stain. The cells are then visualized following excitation under standard 488 nm wavelength light. The ratios of Alexa Flour 488/DAPI in wild-type and CDKN2B/A gene-amended cell populations are quantified and compared to assess the proliferative population ratio within each cell group. Positive Alexa Fluor 488 signal in both total cell number per population treatment and measured over cell passage number indicates DNA replication has occurred and is a direct measure of cells actively proliferating. To quantify and track relative maintenance of proliferative capacity during serial passage, this procedure is repeated during successive population doublings until at least one of the cell populations ceases to proliferate and has become senescent.

### (G) Ectopic Plasmid DNA Transfection Method

To express ectopically, a plasmid DNA construct containing either the TERT or CDK4 regulatory and coding sequences species-specific to the metazoan under study (i.e., the “gene of interest”) is generated using commercial plasmid production services as well as a unique eukaryotic antibiotic resistance gene (i.e. unique to other resident drug-selectable markers). For the intended use described here, plasmid DNA contains a standard vector backbone containing the necessary regulatory elements for amplification in prokaryotic systems.

Each plasmid contains the regulatory and coding regions necessary to express either the TERT or CDK4 protein constitutively within each target cell population. Delivery of the plasmid DNA constructs is mediated by a non-liposomal DNA complex-forming transfection reagent (Fugene HD, Promega) delivered directly the primary cell isolations in suspension and during adhesion. The plasmid contains the regulatory sequences necessary to promote expression of both the gene of interest and the gene encoding the eukaryotic antibiotic resistance. Transfection is performed by using modification of the Grunwald and Speer (2007) protocol for targeted alteration of primary human skeletal muscle myoblasts (Reference Biochemica March 2007, pages 26-27). The prepared plasmid DNA is restriction enzyme digested upstream of the 5′ regulatory components using a unique restriction site. To isolate the cut plasmid from uncut or mis-cut plasmid, a 0.8% agarose gel is run at 10 W/cm until resolved. If all plasmid DNA sampled is linearized appropriately, the cut plasmid is then diluted to a working concentration of 1 μg/μL in sterile, deionized water. 2 μg of total DNA is diluted to a concentration of 1 μg/50 μL in sterile deionized water. Fugene HD is added to the DNA mixture at a ratio of 6:2, (Fugene HD reagent (in μL): total DNA(in μg)) and mixed well. The mixture is incubated at room temperature for fifteen minutes to promote complexation with DNA. The incubated DNA mixture is diluted by adding an equal volume of sterile, deionized water. This entire reaction mixture is then added dropwise to cells in suspension (see below). The cells and DNA reaction mixture is incubated at 37° C. under 5% CO₂, 5% O₂ atmospheric conditions for 48 hours.

(H) Clonal Selection of Cells Transfected with Ectopic DNA

Cell populations are selected via antibiotic drug selection and survival: Cells incubated in the Fugene HD: linearized TERT or CDK4 plasmid DNA sequence have their media removed, are washed in 1×PBS, and fresh proliferation medium is applied. A lethal dosage of antibiotic is then applied to the culture medium to initiate cellular clone selection. The cells are incubated under lethal antibiotic drug exposure at 37° C. under 5% O₂, 5% CO₂ for 14 days. Cells culture medium is monitored and replaced at regular intervals to remove cellular detritus and debris due to drug-induced mortality. Non-morbid, proliferating cell colonies emerging from single cells harboring the antibiotic resistance gene carried on the transfected plasmid are picked using a sterile p200 pipette tip and transferred to a 0.1% gelatin coated well of a 96 well plate. 200 μL of proliferation medium containing the lethal antibiotic concentration are then reapplied as before to maintain constant selection pressure. The cells are incubated at 37° C. under 5% O₂, 5% CO₂ atmospheric conditions until colonies have emerged. Established and proliferating colonies are then dissociated into single cells and expanded into larger-surface area gelatin-coated wells until confluent within 6 wells of a 6 well plate. One confluent well of each 6 well plate of each surviving, expanded cell population targeted by the gene of interest-containing plasmid is then subjected to total genomic DNA isolation. Total cellular genomic DNA is isolated and quantified. Total genomic DNA is then subjected to PCR amplification of the gene of interest's coding sequence and run on a 0.8% agarose gel at 10 W/cm until resolved. These PCR products are then extracted from the agarose gel and submitted for Sanger sequencing. Sequence tracings are compared to wild-type, untreated parental cell populations as well as in silico design of the species-specific gene of interest sequence. Cell populations demonstrating gene of interest genomic sequences that match the in silico prediction are then evaluated for functional and phenotypic characteristics.

### (I) Telomerase Function Validation Assay Using TRAP

Functional evaluation of the TERT expression product is achieved using a standard TRAP assay. TRAP, Telomerase Repeated Amplification Protocol, is performed in the following manner Clonally isolated cell populations determined to possess the TERT construct are plated at equal concentrations alongside cells not containing the TERT construct (untreated cell comparator) and incubated until 80% confluence at 37° C. under 5% CO₂, 5% O₂ atmospheric conditions. Cells are then washed, collected, pelleted, and lysed to collect intracellular protein fraction as well as the genomic DNA. Using a commercially available TRAP assay kit (TRAPeze Telomerase Detection Kit, EMD Millipore) containing the necessary reagents and buffers, PCR amplification is performed on the genomic DNA. The result is run on a non-denaturing acrylamide gel to resolve banding patterns indicative of the presence of an active telomerase (i.e., the pattern of ladder banding that positively correlates with increased active telomerase or its absence).

### CONCLUSION

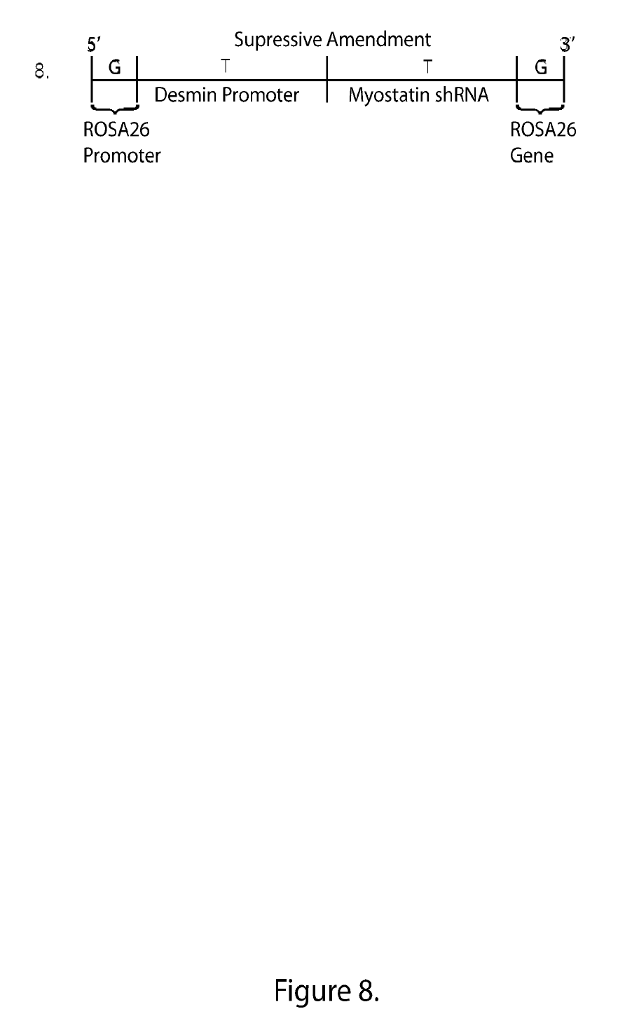

The invention is not limited by the specific techniques used to decouple of retinoblastoma protein suppression of cell division cycle advancement during replicative senescence. In addition to these specific techniques named herein, this suppression can be achieved by other techniques. Examples of such techniques include, but are not limited to the following: (1) Silencing of RNA translation by short-hairpin RNA (FIG. 8); (2) Modulation of endogenous gene expression by guide RNA-targeted nuclease-deficient Cas9 (FIGS. 5 & 6); (3) Lentivirus vector, retrovirus vector, transfected DNA or homologous recombination-directed genetic engineering; (4) Direct targeting inhibition of pRB function or gene expression by methods modeled in FIGS. 4, 6, 7, and 8 to disrupt gene expression from the RB1 locus; (5) Ectopic overexpression of wild-type cyclin-dependent kinase homologs such as CDK6 and CDK2, as well as mutant CDK homologs refractory to CM inhibition, such as CDK4R24C (Wölfel et. al., 1995; Science Vol. 269 pp 1281-1284); (6) Simultaneous disruption of both p15 and p16 function in cells whose species identity features both transcripts; and (7) Disruption of retinoblastoma family protein function using a virus protein targeting retinoblastoma family proteins containing a motif homologous to a pentapeptide motif containing Leucine-X-Cysteine-X-Glutamic Acid.

Though one embodiment of this process results in cultivation of animal biomass for dietary consumption, other applications include bioprocesses for production of textiles such as leather; and medical uses such as therapeutic tissues and vaccines. In addition, given the rapidly increasing global demands for meat, the resource requirements and ecological impacts of meat consumption, clonal, fully myogenic and immortalized livestock cell lines could provide valuable resource in understanding the mechanisms of skeletal muscle development in livestock, improving livestock weight gain, veterinary and other veterinary applications. It should also be understood that the uses described within the scope of this invention encompass, do not exclude, and are not restricted from: (1) applications for cell lines whose genetic identity does not originate from animal species prevalent in agriculture; and (2) the production of animal biomass from ontological cell lineages of tissues other than skeletal muscle.

The taxonomic scope of the present invention and process encompasses species accepted as poultry, including, but not limited to, Gallus gallus, Meleagris gallopavo and Anas platyrhynchos, species accepted as livestock, including, but not limited to, Bos taurus, Sus scrofa and Ovis aries, and species accepted as seafood, including, but not limited to, Salmo salar, Thunnus thynnus, Gadus morhua, Homarus americanus and Litopenaeus setiferus.

What is claimed is:

1. A process for extending the replicative capacity of a metazoan somatic cell population comprising:
decoupling retinoblastoma protein inhibition of cell division cycle advancement during replicative senescence by abrogating cyclin-dependent kinase inhibitor (“CKI”)-mediated stabilization of a retinoblastoma protein using genetic amendment;
maintaining telomerase activity by transducing the metazoan somatic cell population with a genetic construct (SEQ ID NO 11) directing ectopic expression of functional telomere reverse transcriptase (“TERT”) protein;
maintaining a bank of cells that is a master cell bank having the genetic amendment and ectopic expression of TERT protein;
cultivating cells from the master cell bank in an ex vivo milieu that is a cultivated cell biomass; and
harvesting the cultivated cell biomass for dietary consumption.

2. The process of claim 1, wherein the genetic amendment comprises inactivating a p15 protein by genetic amendment of a CDKN2B gene (NCBI Gene ID: 395076) in the metazoan somatic cell population to abolish retinoblastoma protein inhibition of the cell cycle during replicative senescence.

3. The process of claim 2, wherein the genetic amendment is a mutation of a conserved nucleotide sequence in exon one of the CDKN2B gene.

4. The process of claim 3, wherein the genetic amendment is an insertion mutation made using guide RNAs, said guide RNAs selected from the group consisting of guide RNAs (SEQ ID Nos. 1, 2, 3, 4 and 5) targeting exon one of the CDKN2B gene and is created using clustered regularly-interspaced short palindromic repeats-Cas9 (“CRISPR/Cas9”).

5. The process of claim 3, wherein the genetic amendment is a deletion mutation made using guide RNAs, said guide RNAs selected from the group consisting of guide RNAs (SEQ ID Nos. 1, 2, 3, 4 and 5) targeting exon one of the CDKN2B gene and is created using CRISPR/Cas9.

6. The process of claim 2, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

7. The process of claim 2, wherein the metazoan somatic cell population species identity is Gallus gallus and the metazoan somatic cell population lineage is skeletal muscle.

8. The process of claim 1, wherein the genetic amendment comprises inactivating a p16 protein by genetic amendment of a CDKN2A gene (NCBI Gene ID: 616369) in the metazoan somatic cell population to abolish retinoblastoma protein inhibition of the cell cycle during replicative senescence.

9. The process of claim 8, wherein the genetic amendment is a mutation of a conserved nucleotide sequence in exon two of the CDKN2A gene.

10. The process of claim 9, wherein the genetic amendment is an insertion mutation made using guide RNAs targeting exon two of the CDKN2A gene and is created using CRISPR/Cas9.

11. The process of claim 9, wherein the genetic amendment is a deletion mutation made using guide RNAs said guide RNAs selected from the group consisting of guide RNAs (SEQ ID Nos. 8, 9 and 10) targeting exon two of the CDKN2A and is created using CRISPR/Cas9.

12. The process of claim 8, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

13. The process of claim 8, wherein the metazoan somatic cell population species identity is Bos taurus and the metazoan somatic cell population lineage is skeletal muscle.

14. The process of claim 1, wherein the genetic amendment further comprises modifying the cell population with a genetic construct (SEQ ID NO 12) that directs ectopic expression of a cyclin-dependent kinase 4 (“CDK4”) protein homolog, from a CDK4 gene (NCBI Gene ID: 510618).

15. The process of claim 14, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

16. The process of claim 14, wherein the metazoan somatic cell population species identity is Gallus gallus and the metazoan somatic cell population lineage is skeletal muscle.

17. The process of claim 14, wherein modifying the metazoan cell population is with a genetic construct (SEQ ID NO 12) directing expression of a CDK4 protein homolog from a Bos taurus CDK4 gene.

18. A clonal cell line of a metazoan somatic cell population derived by a process comprising:
decoupling retinoblastoma protein inhibition of cell division cycle advancement during replicative senescence by abrogating CM-mediated stabilization of a retinoblastoma protein using genetic amendment;
maintaining telomerase activity by transducing the metazoan somatic cell population with a genetic construct (SEQ ID NO 11) directing ectopic expression of functional TERT protein;
maintaining a bank of cells that is a master cell bank having the genetic amendment and ectopic expression of TERT protein;
cultivating cells from the master cell bank in an ex vivo milieu that is a cultivated cell biomass;
harvesting the cultivated cell biomass for dietary consumption; and
wherein the clonal cell line has indefinite replicative capacity for scalable applications in the industrial production.

19. The clonal cell line of claim 18, wherein the genetic amendment comprises inactivating a p15 protein by genetic amendment of a CDKN2B gene (NCBI Gene ID: 395076) in the metazoan somatic cell population to abolish retinoblastoma protein inhibition of the cell cycle during replicative senescence.

20. The clonal cell line of claim 19, wherein the genetic amendment is a mutation of a conserved nucleotide sequence in exon one of the CDKN2B gene.

21. The clonal cell line of claim 20, wherein the genetic amendment is an insertion mutation made using guide RNAs said guide RNAs selected from the group consisting of (SEQ ID Nos. 1, 2, 3, 4 and 5) targeting exon one of the CDKN2B gene and is created using CRISPR/Cas9.

22. The clonal cell line of claim 20, wherein the genetic amendment is a deletion mutation made using guide RNAs said guide RNAs selected from the group consisting of (SEQ ID Nos. 1, 2, 3, 4 and 5) targeting exon one of the CDKN2B gene and is created using CRISPR/Cas9.

23. The clonal cell line of claim 18, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

24. The clonal cell line of claim 19, wherein the metazoan somatic cell population species identity is Gallus gallus and the metazoan somatic cell population lineage is skeletal muscle.

25. The process of claim 18, wherein the genetic amendment comprises inactivating a p16 protein by genetic amendment of a CDKN2A gene (NCBI Gene ID: 616369) in the metazoan somatic cell population to abolish retinoblastoma protein inhibition of the cell cycle during replicative senescence.

26. The clonal cell line of claim 25, wherein the genetic amendment is a mutation of a conserved nucleotide sequence in exon two of the CDKN2A.

27. The clonal cell line of claim 26, wherein the genetic amendment is an insertion mutation made using guide RNAs said guide RNAs selected from the group consisting of (SEQ ID NO 8-10) targeting exon two of the CDKN2A and is created using CRISPR/Cas9.

28. The clonal cell line of claim 26, wherein the genetic amendment is a deletion mutation made using guide RNAs said guide RNAs selected from the group consisting of (SEQ ID NO 8-10) targeting exon two of the CDKN2A and is created using CRISPR/Cas9.

29. The clonal cell line of claim 25, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

30. The clonal cell line of claim 25, wherein the metazoan somatic cell population species identity is Bos taurus and the metazoan somatic cell population lineage is skeletal muscle.

31. The clonal cell line of claim 18, wherein the genetic amendment further comprises modifying the cell population with a genetic construct that directs ectopic expression of a CDK4 protein homolog, from a CDK4 gene (NCBI Gene ID: 510618).

32. The clonal cell line of claim 31, wherein cultivating cells from the master cell bank further comprises:
expanding selected cell populations from the master cell bank;
cryopreserving and storing expanded cell populations in a master cell bank stock inventory;
seeding and cultivating cells from the master cell bank stock inventory in an ex vivo milieu; and
harvesting cultivated cell biomass for dietary consumption.

33. The clonal cell line of claim 31, wherein the metazoan somatic cell population species identity is Gallus gallus and the metazoan somatic cell population lineage is skeletal muscle.

34. The clonal cell line of claim 31, wherein modifying the metazoan cell population is with a genetic construct (SEQ ID NO 12) directing expression of a CDK4 protein homolog from a Bos taurus CDK4 gene.
